(19) United States
(12) Patent Application Publication
(10) Pub. No.: US 2026/0209342 A1
(43) Pub. Date: Jul. 23, 2026

(54) BIOMARKER FOR CANCER TREATMENT USING ANTI-CLAUDIN-1 ANTIBODIES

(71) Applicant: Alentis Therapeutics AG, Allschwil (CH)

(72) Inventors: Alberto Toso, Basel (CH); Tina Zimmermann, Allschwil (CH)

(21) Appl. No.: 19/157,826

(22) PCT Filed: Mar. 22, 2024
(86) PCT No.: PCT/EP2024/057815

§ 371 Date: Aug. 19, 2025

Related U.S. Application Data
(60) Provisional application No. 63/491,860, filed on Mar. 23, 2023.

(51) Int. Cl. C07K 16/28; A61K 39/00; A61P 35/00; G01N 33/68

(52) U.S. Cl. CPC C07K 16/28; A61P 35/00; G01N 33/6887; A61K 2039/54; A61K 2039/545; C07K 2317/14; C07K 2317/24; C07K 2317/565; G01N 2333/78; G01N 2800/52

(57) ABSTRACT

According to various aspects of this disclosure, the present disclosure relates to a method of treating cancer comprising (a) administering an anti-Claudin-1 antibody to a subject; (b) determining a level of PRO-C3 in a test sample from the subject; and (c) comparing the level of PRO-C3 in the test sample to a level of PRO-C3 in a control sample. In some aspects, if the level of PRO-C3 in the test sample is not decreased relative to the level of PRO-C3 in the control sample, then the dose of the anti-Claudin-1 antibody being administered to the subject is increased. In some aspects, if the level of PRO-C3 in the test sample is decreased relative to the control sample, then the dose of the anti-Claudin-1 antibody being administered to the subject is not increased.

### CROSS-REFERENCE TO RELATED APPLICATIONS

The present application claims the benefit of U.S. Provisional Patent Application No. 63/491,860, filed Mar. 23, 2023, which is incorporated herein by reference in its entirety.

### INCORPORATION-BY-REFERENCE OF SEQUENCE LISTING

The Sequence Listing XML associated with this application is provided electronically in XML file format and is hereby incorporated by reference into the specification. The name of the XML file containing the Sequence Listing XML is “ALNT-012/001WO_SeqList.xml”. The XML file is 20,878 bytes, created on Mar. 7, 2024 and is being submitted electronically via USPTO Patent Center.

### FIELD

According to various aspects of this disclosure, the present disclosure relates to methods of treatment comprising administration of anti-Claudin-1 antibodies and quantification of N-terminal propeptide of type III collagen (“PRO-C3”) or granzyme B degraded type IV collagen products (“C4G”).

### BACKGROUND

Anti-Claudin-1 antibodies have been used to treat an array of diseases, such as hepatocellular carcinoma (e.g., U.S. Pat. No. 10,815,298), non-alcoholic fatty liver disease (e.g., U.S. Pat. No. 10,927,170), and fibrotic diseases such as renal fibrosis, pulmonary fibrosis, and skin fibrosis (e.g., WO 2021/094469 A1).

Claudin 1 (CLDN1) is a protein confined within the normal epithelial tight junctions of different tissues. Upon malignant transformation, CLDN1 is overexpressed and epitopes become exposed outside the tight junctions (non-junctional CLDN1).

A need exists to identify biomarkers that will improve treatment with anti-Claudin-1 antibodies (e.g., treatment of cancer).

### SUMMARY

Certain aspects of the disclosure provide a method of treating cancer comprising: (a) administering an anti-Claudin-1 antibody to a subject; (b) determining a level of PRO-C3 in a test sample from the subject; and (c) comparing the level of PRO-C3 in the test sample to a level of PRO-C3 in a control sample. In some aspects, if the level of PRO-C3 in the test sample is not decreased relative to the level of PRO-C3 in the control sample, then the dose of the anti-Claudin-1 antibody being administered to the subject is increased. In some aspects, if the level of PRO-C3 in the test sample is decreased relative to the control sample, then the dose of the anti-Claudin-1 antibody being administered to the subject is not increased.

Certain aspects of the disclosure provide a method of treating cancer in a subject, in which the subject has been determined to have a level of PRO-C3 in a test sample from the subject that is not decreased relative to a level of PRO-C3 in a control sample, the method comprising administering an increased dose of the anti-Claudin-1 treatment to the subject. In some aspects, the subject has previously been administered an anti-Claudin-1 treatment.

Certain aspects of the disclosure provide a method of monitoring cancer progression in a subject comprising: (a) quantifying a level of PRO-C3 in a test sample from the subject, wherein the subject has previously been administered an anti-Claudin-1 treatment; and (b) comparing the level of PRO-C3 in the test sample to a level of PRO-C3 in a control sample. In some aspects, a level of PRO-C3 in the test sample that is not decreased relative to the level of PRO-C3 in the control sample indicates that the subject is susceptible to an increased dose of the anti-Claudin-1 treatment.

Certain aspects of the disclosure provide a method of monitoring cancer progression in a subject comprising: (a) determining a level of PRO-C3 in a test sample from the subject, wherein the subject has previously been administered an anti-Claudin-1 antibody; and (b) comparing the level of PRO-C3 in the test sample to a level of PRO-C3 in a control sample. In some aspects, a level of PRO-C3 in the test sample that is not decreased relative to the level of PRO-C3 in the control sample indicates that the subject is susceptible to an increased dose of the anti-Claudin-1 antibody.

Certain aspects of the disclosure provide a method for treating a subject suffering from cancer, the method comprising the steps of: determining whether the subject has decreased levels of PRO-C3 by obtaining a test sample from the subject and comparing the level of PROC3 in the test sample to a control sample, wherein the subject has previously been administered a dose of an anti-Claudin-1 antibody. In some aspects, if the subject has decreased levels of PROC3 in the test sample relative to the control sample, then the subject is not administered an increased dose of the anti-Claudin-1 antibody. In some aspects, if the subject does not have decreased levels of PRO-C3 in the test sample relative to the control sample, then the subject is administered an increased dose of the anti-Claudin-1 antibody.

Certain aspects of the disclosure provide a method for designing a customized therapy for a subject suffering from cancer which comprises (a) quantifying the level of PRO-C3 in a test sample from the subject; (b) comparing the level of PRO-C3 in the test sample obtained in step (a) with the level of PRO-C3 in a control sample; (c) determining that the subject has a not decreased level of PRO-C3 with respect to the level of PRO-C3 in the control sample; and (d) administering an increased dose of the anti-Claudin-1 antibody.

In some aspects, the subject has previously been administered a dose of an anti-Claudin-1 antibody. In some aspects, if the level of PRO-C3 in the test sample obtained in step (a) is not decreased relative to the level of PRO-C3 in the control sample, then the subject is susceptible to receive an increased dose of the anti-Claudin-1 antibody.

Certain aspects of the disclosure provide a method of classifying a subject suffering from cancer into a cohort and treating said subject, comprising (a) quantifying the level of PROC3 in a test sample from the subject; (b) comparing the level of PRO-C3 in the test sample obtained in step (a) with the level of PRO-C3 in a control sample; (c) determining that the subject has a not decreased level of PRO-C3 with respect to the level of PRO-C3 in the control sample; (d) classifying said subject into a cohort based on the level of PRO-C3 in the test sample relative to the control sample; and (e) administering an increased dose of the anti-Claudin-1 antibody. In some aspects, the subject has previously been administered a dose of an anti-Claudin-1 antibody. In some aspects, if the level of PRO-C3 in the test sample obtained in step (a) is not decreased relative to the level of PRO-C3 in the control sample, then the subject is susceptible to receive an increased dose of the anti-Claudin-1 antibody.

Certain aspects of the disclosure provide a method of treating cancer comprising: (a) administering an anti-Claudin-1 antibody to a subject; (b) determining a level of C4G in a test sample from the subject; and (c) comparing the level of C4G in the test sample to a level of C4G in a control sample. In some aspects, if the level of C4G in the test sample is not increased relative to the level of C4G in the control sample, then the dose of the anti-Claudin-1 antibody being administered to the subject is increased. In some aspects, if the level of C4G in the test sample is increased relative to the control sample, then the dose of the anti-Claudin-1 antibody being administered to the subject is not increased.

Certain aspects of the disclosure provide a method of treating cancer in a subject, in which the subject has been determined to have a level of C4G in a test sample from the subject that is not increased relative to a level of C4G in a control sample, the method comprising administering an increased dose of the anti-Claudin-1 treatment to the subject. In some aspects, the subject has previously been administered an anti-Claudin-1 treatment.

Certain aspects of the disclosure provide a method of monitoring cancer progression in a subject comprising: (a) quantifying a level of C4G in a test sample from the subject, wherein the subject has previously been administered an anti-Claudin-1 treatment; and (b) comparing the level of C4G in the test sample to a level of C4G in a control sample; wherein a level of C4G in the test sample that is not increased relative to the level of C4G in the control sample indicates that the subject is susceptible to an increased dose of the anti-Claudin-1 treatment.

Certain aspects of the disclosure provide a method of monitoring cancer progression in a subject comprising: (a) determining a level of C4G in a test sample from the subject, wherein the subject has previously been administered an anti-Claudin-1 antibody; and (b) comparing the level of C4G in the test sample to a level of C4G in a control sample. In some aspects, a level of C4G in the test sample that is not increased relative to the level of C4G in the control sample indicates that the subject is susceptible to an increased dose of the anti-Claudin-1 antibody.

Certain aspects of the disclosure provide a method for treating a subject suffering from cancer, the method comprising the steps of: determining whether the subject has increased levels of C4G by obtaining a test sample from the subject and comparing the level of C4G in the test sample to a control sample. In some aspects, the subject has previously been administered a dose of an anti-Claudin-1 antibody. In some aspects, if the subject has increased levels of C4G in the test sample relative to the control sample, then the subject is not administered an increased dose of the anti-Claudin-1 antibody. In some aspects, if the subject does not have increased levels of C4G in the test sample relative to the control sample, then the subject is administered an increased dose of the anti-Claudin-1 antibody.

Certain aspects of the disclosure provide a method for designing a customized therapy for a subject suffering from cancer which comprises (a) quantifying the level of C4G in a test sample from the subject; (b) comparing the level of C4G in the test sample obtained in step (a) with the level of C4G in a control sample, (c) determining that the subject has a not increased level of C4G with respect to the level of C4G in the control sample; and (d) administering an increased dose of the anti-Claudin-1 antibody. In some aspects, the subject has previously been administered a dose of an anti-Claudin-1 antibody. In some aspects, if the level of C4G in the test sample obtained in step (a) is not increased relative to the level of C4G in the control sample, then the subject is susceptible to receive an increased dose of the anti-Claudin-1 antibody.

Certain aspects of the disclosure provide a method of classifying a subject suffering from cancer into a cohort and treating said subject, comprising (a) quantifying the level of C4G in a test sample from the subject; (b) comparing the level of C4G in the test sample obtained in step (a) with the level of C4G in a control sample; (c) determining that the subject has a not increased level of C4G with respect to the level of C4G in the control sample; (d) classifying said subject into a cohort based on the level of C4G in the test sample relative to the control sample; and (e) administering an increased dose of the anti-Claudin-1 antibody. In some aspects, the subject has previously been administered a dose of an anti-Claudin-1 antibody. In some aspects, if the level of C4G in the test sample obtained in step (a) is not increased relative to the level of C4G in the control sample, then the subject is susceptible to receive an increased dose of the anti-Claudin-1 antibody.

In some aspects, if the subject is susceptible to an increased dose of the anti-Claudin-1 antibody, then the anti-Claudin-1 antibody is administered to the subject.

In some aspects, the anti-Claudin-1 treatment is an anti-Claudin-1 antibody.

In some aspects, the anti-Claudin-1 antibody is administered intratumorally, intravenously, intraperitoneally, intramuscularly, intrathecally or subcutaneously.

In some aspects, the cancer is selected from the group consisting of a head and neck cancer (e.g., Head and Neck Squamous Cell Carcinoma), a lung cancer, a breast cancer, a melanoma, a colorectal cancer, a pancreatic cancer, an esophageal cancer, a cholangiocarcinoma, and a hepatocellular carcinoma.

In some aspects, the control sample is a sample from the subject prior to being administered an anti-Claudin-1 antibody or an anti-Claudin-1 treatment.

In some aspects, the level of PRO-C3 in the control sample is about 10 ng/ml to about 20 ng/mL.

In some aspects, the level of C4G in the control sample is about 30 ng/mL to about 40 ng/mL.

In some aspects, the anti-Claudin-1 antibody is a monoclonal antibody comprising the six complementarity determining regions (CDRs) of an anti-Claudin-1 monoclonal antibody secreted by a hybridoma cell line deposited at the DSMZ on Jul. 29, 2008 under an Accession Number DSM ACC2938.

In some aspects, the anti-Claudin-1 antibody is humanized.

In some aspects, the anti-Claudin-1 antibody comprises a VH comprising the amino acid sequence set forth in SEQ ID NO: 3 or SEQ ID NO: 13.

In some aspects, the anti-Claudin-1 antibody comprises a VL comprising the amino acid sequence set forth in SEQ ID NO: 4 or SEQ ID NO: 14.

In some aspects, the anti-Claudin-1 antibody comprises a VH comprising the amino acid sequence set forth in SEQ ID NO: 3; and a VL comprising the amino acid sequence set forth in SEQ ID NO: 4.

In some aspects, the anti-Claudin-1 antibody comprises a VH comprising the amino acid sequence set forth in SEQ ID NO: 13; and a VL comprising the amino acid sequence set forth in SEQ ID NO: 14.

In some aspects, the anti-Claudin-1 antibody comprises a complementarity determining region (CDR) H1 comprising the amino acid sequence set forth in SEQ ID NO: 5, a CDR H2 comprising the amino acid sequence set forth in SEQ ID NO: 6, and a CDR H3 comprising the amino acid sequence set forth in SEQ ID NO: 7.

In some aspects, the anti-Claudin-1 antibody comprises a complementarity determining region (CDR) L1 comprising the amino acid sequence set forth in SEQ ID NO: 8, a CDR L2 comprising the amino acid sequence GA, and a CDR L3 comprising the amino acid sequence set forth in SEQ ID NO: 10.

In some aspects, the anti-Claudin-1 antibody comprises a heavy chain comprising the amino acid sequence set forth in SEQ ID NO: 1.

In some aspects, the anti-Claudin-1 antibody comprises a light chain comprising the amino acid sequence set forth in SEQ ID NO: 2.

### DESCRIPTION OF FIGURES

FIG. 1 shows CLDN1 mRNA expression in solid tumors.

FIG. 2 shows anti-CLDN1 antibody treatment of head and neck squamous cell carcinoma (HNSCC) in a patient-derived xenograft model relative to IgG1 control as measured by tumor volume (mm3).

FIG. 3A shows PRO-C3 levels (ng/mL) after treatment with anti-CLDN1 antibody or IgG1 in a patient-derived xenograft model of head and neck squamous cell carcinoma (HNSCC).

FIG. 3B shows C4G levels (ng/ml) after treatment with anti-CLDN1 antibody or IgG1 in a patient-derived xenograft model of head and neck squamous cell carcinoma (HNSCC).

FIG. 4A shows ADCC measured in vitro between anti-CLDN1 antibodies with a WT IgG1 Fc or a Fc mutant relative to IgG1 control.

FIG. 4B shows tumor growth inhibition (TGI) in a CAL27 HNSCC CDX model after treatment with IgG1 control, anti-CLDN1 antibody (WT IgG1 Fc), or anti-CLDN1 antibody (Fc mutant).

### DETAILED DESCRIPTION

In some aspects, provided herein is a method of treating cancer comprising (a) administering an anti-Claudin-1 antibody to a subject; (b) determining a level of PRO-C3 in a test sample from the subject; and (c) comparing the level of PRO-C3 in the test sample to a level of PRO-C3 in a control sample. In some aspects, if the level of PRO-C3 in the test sample is not decreased relative to the level of PRO-C3 in the control sample, then the dose of the anti-Claudin-1 antibody being administered to the subject is increased. In some aspects, if the level of PRO-C3 in the test sample is decreased relative to the control sample, then the dose of the anti-Claudin-1 antibody being administered to the subject is not increased.

In some aspects, provided herein is a method of treating cancer in a subject that has been determined to have a level of PRO-C3 in a test sample from the subject that is not decreased relative to a level of PRO-C3 in a control sample. In some aspects, the subject has previously been administered an anti-Claudin-1 treatment, the method comprising administering an increased dose of the anti-Claudin-1 treatment to the subject.

In some aspects, provided herein is a method of monitoring cancer progression in a subject comprising (a) quantifying a level of PRO-C3 in a test sample from the subject, wherein the subject has previously been administered an anti-Claudin-1 treatment; and (b) comparing the level of PRO-C3 in the test sample to a level of PRO-C3 in a control sample. In some aspects, a level of PRO-C3 in the test sample that is not decreased relative to the level of PRO-C3 in the control sample indicates that the subject is susceptible to an increased dose of the anti-Claudin-1 treatment.

In some aspects, provided herein is a method of monitoring cancer progression in a subject comprising (a) determining a level of PRO-C3 in a test sample from the subject, wherein the subject has previously been administered an anti-Claudin-1 antibody; and (b) comparing the level of PRO-C3 in the test sample to a level of PRO-C3 in a control sample. In some aspects, a level of PRO-C3 in the test sample that is not decreased relative to the level of PRO-C3 in the control sample indicates that the subject is susceptible to an increased dose of the anti-Claudin-1 antibody.

In some aspects, provided herein is a method for treating a subject suffering from cancer, the method comprising the steps of determining whether the subject has decreased levels of PRO-C3 by obtaining a test sample from the subject and comparing the level of PRO-C3 in the test sample to a control sample. In some aspects, the subject has previously been administered a dose of an anti-Claudin-1 antibody. In some aspects, if the subject has decreased levels of PRO-C3 in the test sample relative to the control sample, then the subject is not administered an increased dose of the anti-Claudin-1 antibody. In some aspects, if the subject does not have decreased levels of PRO-C3 in the test sample relative to the control sample, then the subject is administered an increased dose of the anti-Claudin-1 antibody.

In some aspects, provided herein is a method for designing a customized therapy for a subject suffering from cancer which comprises (a) quantifying the level of PRO-C3 in a test sample from the subject; (b) comparing the level of PRO-C3 in the test sample obtained in step (a) with the level of PRO-C3 in a control sample, (c) determining that the subject has a not decreased level of PRO-C3 with respect to the level of PRO-C3 in the control sample; and (d) administering an increased dose of the anti-Claudin-1 antibody. In some aspects, wherein if the level of PRO-C3 in the test sample obtained in step (a) is not decreased relative to the level of PRO-C3 in the control sample, then the subject is susceptible to receive an increased dose of the anti-Claudin-1 antibody. In some aspects, the subject has previously been administered a dose of an anti-Claudin-1 antibody.

In some aspects, provided herein is a method of classifying a subject suffering from cancer into a cohort and treating said subject, comprising (a) quantifying the level of PRO-C3 in a test sample from the subject; (b) comparing the level of PRO-C3 in the test sample obtained in step (a) with the level of PRO-C3 in a control sample; (c) determining that the subject has a not decreased level of PRO-C3 with respect to the level of PRO-C3 in the control sample; (d) classifying said subject into a cohort based on the level of PRO-C3 in the test sample relative to the control sample; and (e) administering an increased dose of the anti-Claudin-1 antibody. In some aspects, the subject has previously been administered a dose of an anti-Claudin-1 antibody. In some aspects, if the level of PRO-C3 in the test sample obtained in step (a) is not decreased relative to the level of PRO-C3 in the control sample, then the subject is susceptible to receive an increased dose of the anti-Claudin-1 antibody.

In some aspects, provided herein is a method of treating cancer comprising: (a) administering an anti-Claudin-1 antibody to a subject; (b) determining a level of C4G in a test sample from the subject; and (c) comparing the level of C4G in the test sample to a level of C4G in a control sample. In some aspects, if the level of C4G in the test sample is not increased relative to the level of C4G in the control sample, then the dose of the anti-Claudin-1 antibody being administered to the subject is increased. In some aspects, if the level of C4G in the test sample is increased relative to the control sample, then the dose of the anti-Claudin-1 antibody being administered to the subject is not increased.

In some aspects, provided herein is a method of treating cancer in a subject that has been determined to have a level of C4G in a test sample from the subject that is not increased relative to a level of C4G in a control sample. In some aspects, the subject has previously been administered an anti-Claudin-1 treatment, the method comprising administering an increased dose of the anti-Claudin-1 treatment to the subject.

In some aspects, provided herein is a method of monitoring cancer progression in a subject comprising (a) quantifying a level of C4G in a test sample from the subject, wherein the subject has previously been administered an anti-Claudin-1 treatment; and (b) comparing the level of C4G in the test sample to a level of C4G in a control sample. In some aspects, a level of C4G in the test sample that is not increased relative to the level of C4G in the control sample indicates that the subject is susceptible to an increased dose of the anti-Claudin-1 treatment.

In some aspects, provided herein is a method of monitoring cancer progression in a subject comprising (a) determining a level of C4G in a test sample from the subject; and (b) comparing the level of C4G in the test sample to a level of C4G in a control sample. In some aspects, the subject has previously been administered an anti-Claudin-1 antibody. In some aspects, a level of C4G in the test sample that is not increased relative to the level of C4G in the control sample indicates that the subject is susceptible to an increased dose of the anti-Claudin-1 antibody.

In some aspects, provided herein is a method for treating a subject suffering from cancer, the method comprising the steps of determining whether the subject has increased levels of C4G by obtaining a test sample from the subject and comparing the level of C4G in the test sample to a control sample. In some aspects, the subject has previously been administered a dose of an anti-Claudin-1 antibody. In some aspects, if the subject has increased levels of C4G in the test sample relative to the control sample, then the subject is not administered an increased dose of the anti-Claudin-1 antibody. In some aspects, if the subject does not have increased levels of C4G in the test sample relative to the control sample, then the subject is administered an increased dose of the anti-Claudin-1 antibody.

In some aspects, provided herein is a method for designing a customized therapy for a subject suffering from cancer which comprises (a) quantifying the level of C4G in a test sample from the subject; (b) comparing the level of C4G in the test sample obtained in step (a) with the level of C4G in a control sample; (c) determining that the subject has a not increased level of C4G with respect to the level of C4G in the control sample; and (d) administering an increased dose of the anti-Claudin-1 antibody. In some aspects, the subject has previously been administered a dose of an anti-Claudin-1 antibody. In some aspects, if the level of C4G in the test sample obtained in step (a) is not increased relative to the level of C4G in the control sample, then the subject is susceptible to receive an increased dose of the anti-Claudin-1 antibody.

In some aspects, provided herein is a method of classifying a subject suffering from cancer into a cohort and treating said subject, comprising (a) quantifying the level of C4G in a test sample from the subject; (b) comparing the level of C4G in the test sample obtained in step (a) with the level of C4G in a control sample; (c) determining that the subject has a not increased level of C4G with respect to the level of C4G in the control sample; (d) classifying said subject into a cohort based on the level of C4G in the test sample relative to the control sample; and (e) administering an increased dose of the anti-Claudin-1 antibody. In some aspects, the subject has previously been administered a dose of an anti-Claudin-1 antibody. In some aspects, if the level of C4G in the test sample obtained in step (a) is not increased relative to the level of C4G in the control sample, then the subject is susceptible to receive an increased dose of the anti-Claudin-1 antibody.

### Definitions

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

In order to further define this disclosure, the following terms and definitions are provided.

The singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6, etc., as well as individual numbers within that range, for example, 1, 2, 3, 4, 5, and 6. This applies regardless of the breadth of the range. Numeric ranges recited are inclusive of the numbers defining the range and include each integer within the defined range.

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this disclosure are based upon antibodies, in certain aspects, the polypeptides can occur as single chains or associated chains.

The terms “administer,” “administering,” “administration,” and the like, as used herein, refer to the physical introduction of a composition comprising a therapeutic agent (e.g., combination of an anti-Claudin-1 antibody) to a subject, using any of the various methods and delivery systems known to those skilled in the art. Routes of administration include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Other nonparenteral routes include a topical, epidermal or mucosal route of administration, for example, intranasally, vaginally, rectally, sublingually or topically. Administering can also be performed, for example, once, a plurality of times, and/or over one or more extended periods.

The terms “treat,” “treatment,” or “treating,” as used herein refers to, e.g., the reduction in severity of a disease or condition; the reduction in the duration of a disease course; the amelioration or elimination of one or more symptoms associated with a disease or condition; the provision of beneficial effects to a subject with a disease or condition, without necessarily curing the disease or condition. In one aspect, the term “treating” or “treatment” means treating cancer (e.g. a head and neck cancer (e.g., Head and Neck Squamous Cell Carcinoma), a lung cancer, a breast cancer, a melanoma, a colorectal cancer, a pancreatic cancer, an esophageal cancer, a cholangiocarcinoma, and a hepatocellular carcinoma).

The terms “subject,” “patient,” “individual,” and “host,” and variants thereof are used interchangeably herein and refer to any mammalian subject, including without limitation, humans, domestic animals (e.g., dogs, cats and the like), farm animals (e.g., cows, sheep, pigs, horses and the like), and laboratory animals (e.g., monkey, rats, mice, rabbits, guinea pigs and the like) for whom diagnosis, treatment, or therapy is desired, particularly humans. The methods described herein are applicable to both human therapy and veterinary applications. As used herein, the phrase “subject in need thereof” includes subjects, such as mammalian subjects, that would benefit from administration of a therapeutic agent, e.g., an anti-Claudin-1 antibody.

An “anti-Claudin-1 treatment” as used herein is intended to refer to a therapy that targets Claudin-1, for example, treatment with anti-Claudin-1 antibodies or Claudin-1 targeting CARs described herein. In some embodiments, an anti-Claudin-1 treatment is an anti-Claudin-1 antibody disclosed herein.

It is understood that wherever aspects are described herein with the language “comprising,” otherwise analogous aspects described in terms of “consisting of” and/or “consisting essentially of” are also provided.

As used herein, the terms “derived from” or “derivative” refer to a component that is isolated from or made using a specified molecule, or information (e.g., a nucleic acid sequence) from the specified molecule. For example, a polynucleotide sequence that is derived from another polynucleotide sequence can include a polynucleotide sequence that is identical or substantially similar to the polynucleotide sequence it derives from. In the case of polynucleotides, the derived species can be obtained by, for example, naturally occurring mutagenesis, artificial directed mutagenesis, or artificial random mutagenesis. The mutagenesis used to derive polynucleotides can be intentionally directed or intentionally random, or a mixture of each. The mutagenesis of a polynucleotide to create a different polynucleotide derived from the first polynucleotide can be a random event (e.g., caused by polymerase infidelity) and the identification of the derived polynucleotide can be made by appropriate screening methods known in the art. In some aspects, a polynucleotide sequence that is derived from a first polynucleotide sequence has a sequence identity of at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identity to the first polynucleotide sequence, respectively, wherein the derived polynucleotide sequence retains the biological activity of the original polynucleotide.

The term “human Claudin-1 (or CLDN1)” refers to a protein having the sequence shown in NCBI Accession Number NP_066924.1, or any naturally occurring variants commonly found in HCV permissive human populations.

The term “antibody”, as used herein, refers to any immunoglobulin that contains an antigen binding site that immunospecifically binds an antigen. As such, the term antibody encompasses not only whole antibody molecules, but also antibody fragments as well as variants (including derivatives) of antibodies and of antibody fragments as long as the derivatives and fragments maintain specific binding ability. The term encompasses monoclonal antibodies and polyclonal antibodies. The term also covers any protein having a binding domain, which is homologous or largely homologous to an immunoglobulin-binding domain. These proteins may be derived from natural sources, or partly or wholly synthetically produced. The term “specific binding”, when used in reference to an antibody, refers to an antibody binding to a predetermined antigen. Typically, the antibody binds with an affinity of at least 1×107 M1, and binds to the predetermined antigen with an affinity that is at least two-fold greater than the affinity for binding to a non-specific antigen (e.g., BSA, casein).

The term “monoclonal antibody” or antigen-binding fragment thereof refers to a homogeneous antibody or antigen-binding fragment population involved in the highly specific recognition and binding of a single antigenic determinant, or epitope. This is in contrast to polyclonal antibodies that typically include different antibodies directed against different antigenic determinants. The term “monoclonal antibody” or antigen-binding fragment thereof encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen-recognition site. Furthermore, a “monoclonal antibody” or antigen-binding fragment thereof refers to such antibodies and antigen-binding fragments thereof made in any number of manners including but not limited to by hybridoma, phage selection, recombinant expression, and transgenic animals.

As used herein, the term “humanized antibody” refers to a chimeric antibody comprising amino acid residues from non-human hypervariable regions and amino acid residues from human framework regions (FRs). In particular, a humanized antibody comprises all or substantially all of at least one, typically two, variable domains, in which all or substantially all of the complementarity determining regions (CDRs) are those of a human antibody. A humanized antibody optionally may comprise at least a portion of an antibody constant region derived from a human antibody. A “humanized form” of an antibody, e.g., a non-human antibody, refers to an antibody that has undergone humanization.

The term “effective amount” refers to an amount of an agent that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system (e.g., cancer). An effective amount can be administered in one or more administrations.

The term “PRO-C3” refers to the N-terminal propeptide of type III collagen. Cleavage of the N-terminal propeptide of type III collagen may be mediated by an N-protease. In some aspects, the PRO-C3 is derived from a human COL3A1 homotrimer (e.g., NP_000081.2).

The term “C4G” refers to granzyme B degraded type IV collagen products.

### Anti-Claudin-1 Antibodies

Antibodies directed against human Claudin-1 have been previously described to treat hepatitis C virus infection, hepatocellular carcinoma, and certain fibrotic diseases, such as lung fibrosis (see WO 2010/034812, WO 2016/146809, and WO 2021/094469). Anti-Claudin-1 antibodies that can be used in the practice of the methods disclosed herein include any antibody raised against Claudin-1. Examples are disclosed in WO 2010/034812 and WO 2017/162678, each of which is incorporated herein by reference in its entirety for examples of anti-Claudin-1 antibodies that may be used in the methods described herein.

Other examples of suitable anti-Claudin-1 antibodies include those disclosed in European Patent No. EP 1 167 389, in U.S. Pat. No. 6,627,439, in international patent application published under No. WO 2014/132307, in international patent applications published under No. WO 2015/014659 and No. WO 2015/014357, and in Yamashita et al., J. Pharmacol. Exp. Ther., 2015, 353 (1): 112-118, each of which is incorporated herein by reference in its entirety for examples of anti-Claudin-1 antibodies that may be used in the methods described herein.

Anti-Claudin-1 antibodies suitable for use in the methods disclosed herein may be polyclonal antibodies or monoclonal antibodies.

Anti-Claudin-1 antibodies suitable for use according to the present disclosure may also be “humanized”: sequence differences between rodent antibodies and human sequences can be minimized by replacing residues which differ from those in the human sequences by site-directed mutagenesis of individual residues or by grafting of entire regions or by chemical synthesis. Humanized antibodies can also be produced using recombinant methods. In the humanized form of the antibody, some, most or all of the amino acids outside the CDR regions are replaced with amino acids from human immunoglobulin molecules, while some, most or all amino acids within one or more CDR regions are unchanged. Small additions, deletions, insertions, substitutions or modifications of amino acids are permissible, as long as they do not significantly modify the biological activity of the resulting antibody. Suitable human “replacement” immunoglobulin molecules include IgG1, IgG2, IgG2a, IgG2b, IgG3, IgG4, IgA, IgM, IgD or IgE molecules, and fragments thereof.

Similarly, conservative amino acid substitutions may be introduced into the antibody sequences disclosed herein, as long as these do not significantly modify the biological activity of the resulting antibody. A “conservative amino acid substitution” as used herein, is one in which one amino acid residue is replaced with another amino acid residue are substitutions that change an amino acid to a different amino acid with similar biochemical properties (e.g. charge, hydrophobicity and size). For example, lysine, arginine and histidine have similar properties in that they have a basic sidechain, and aspartic acid and glutamic acid have similar properties in that they have an acidic side chain. In addition, glycine, asparagine, glutamine, serine, threonine, tyrosine, cysteine and tryptophan have similar properties in that they have an uncharged polar sidechain, and alanine, valine, leucine, threonine, isoleucine, proline, phenylalanine and methionine have similar properties in that they have a non-polar sidechain. Also, tyrosine, phenylalanine, tryptophan and histidine have similar properties in that they have an aromatic sidechain. Thus, it will be obvious to those skilled in the art that, even when substitution of amino acid residues in groups showing similar properties as described above occurs; it will show no particular change in the properties. Variants of polypeptides also include additions and deletions to the polypeptide sequences disclosed herein. In addition, variant nucleotide sequences include analogs and derivatives thereof. A variant of the binding proteins disclosed herein include proteins that bind to the same antigen or epitope as the binding proteins.

In some aspects, a humanized anti-Claudin-1 antibody for use according to the present disclosure is one previously described in WO 2017/162678. Exemplary sequences for the antibody or antigen binding fragment provided herein are described in Table 1.

TABLE 1

| | | SEQ ID |
|---|---|---|
| Description | Sequence | NO: |
| H1L1-Heavy | EVQLVESGGGLVKPGGSLRLSCAASGFSFSSYGMN | 1 |
| Chain #1 | WVRQAPGKGLEWVSSISPSGSYFYYADSVKGRFTIS | |
| | RDNAKNSLYLQMNSLRAEDTAVYYCARLPGFNPPF | |
| | DHWGQGTLVTVSSASTKGPSVFPLAPSSKSTSGGTA | |
| | ALGCLVKDYFPEPVTVSWNSGALTSGVHTFPAVLQS | |
| | SGLYSLSSVVTVPSSSLGTQTYICNVNHKPSNTKVDK | |
| | RVEPKSCDKTHTCPPCPAPELLGGPSVFLFPPKPKDT | |
| | LMISRTPEVTCVVVDVSHEDPEVKFNWYVDGVEVH | |
| | NAKTKPREEQYNSTYRVVSVLTVLHQDWLNGKEYK | |
| | CKVSNKALPAPIEKTISKAKGQPREPQVYTLPPSREE | |
| | MTKNQVSLTCLVKGFYPSDIAVEWESNGQPENNYK | |
| | TTPPVLDSDGSFFLYSKLTVDKSRWQQGNVFSCSVM | |
| | HEALHNHYTQKSLSLSPG | |
| H1L1-Light Chain | DIQMTQSPATLSVSPGERATLSCKASQNVGGNVDW | 2 |
| | YQWKPGQAPRLLIYGASNRYTGIPARFRGSGSGTEF | |
| | TLTISSLQSEDFAVYYCLQYKNNPWTFGQGTKVEIK | |
| | RTVAAPSVFIFPPSDEQLKSGTASVVCLLNNFYPREA | |
| | KVQWKVDNALQSGNSQESVTEQDSKDSTYSLSSTLT | |
| | LSKADYEKHKVYACEVTHQGLSSPVTKSFNRGEC | |
| H1L1-VH | EVQLVESGGGLVKPGGSLRLSCAASGFSFSSYGMN | 3 |
| | WVRQAPGKGLEWVSSISPSGSYFYYADSVKGRFTIS | |
| | RDNAKNSLYLQMNSLRAEDTAVYYCARLPGFNPPF | |
| | DHWGQGTLVTVSS | |
| H1L1-VL | DIQMTQSPATLSVSPGERATLSCKASQNVGGNVDW | 4 |
| | YQWKPGQAPRLLIYGASNRYTGIPARFRGSGSGTEF | |
| | TLTISSLQSEDFAVYYCLQYKNNPWTFGQGTKVEIK | |
| H1L1/H3L3-CDR H1 | GFSFSSYG | 5 |
| H1L1/H3L3-CDR H2 | ISPSGSYF | 6 |
| H1L1/H3L3-CDR H3 | ARLPGFNPPFDH | 7 |
| H1L1/H3L3-CDR L1 | QNVGGN | 8 |
| H1L1/H3L3-CDR L2 | GA | |
| H1L1/H3L3-CDR L3 | LQYKNNPWT | 10 |
| H3L3-Heavy Chain | QVQLVESGGGVVQPGRSLRLSCLGSGFSFSSYGMN | 11 |
| | WVRQAPGKGLEWVASISPSGSYFYYADSVKGRFTIS | |
| | RDNSKNTLYLQMTSLRAEDTAIYYCARLPGFNPPFD | |
| | HWGQGTLVTVSSASTKGPSVFPLAPSSKSTSGGTAA | |
| | LGCLVKDYFPEPVTVSWNSGALTSGVHTFPAVLQSS | |
| | GLYSLSSVVTVPSSSLGTQTYICNVNHKPSNTKVDK | |
| | RVEPKSCDKTHTCPPCPAPELLGGPSVFLFPPKPKDT | |
| | LMISRTPEVTCVVVDVSHEDPEVKFNWYVDGVEVH | |
| | NAKTKPREEQYNSTYRVVSVLTVLHQDWLNGKEYK | |
| | CKVSNKALPAPIEKTISKAKGQPREPQVYTLPPSREE | |
| | MTKNQVSLTCLVKGFYPSDIAVEWESNGQPENNYK | |
| | TTPPVLDSDGSFFLYSKLTVDKSRWQQGNVFSCSVM | |
| | HEALHNHYTQKSLSLSPG | |
| H3L3-Light Chain | DIQMTQSPSSLSASVGDRVTITCKASQNVGGNVDWY | 12 |
| | QWKPGKAPKLLIYGASNRYTGVPDRFRGSGSGTDFT | |
| | LTISSLQPEDVATYYCLQYKNNPWTFGGGTKVEIKR | |
| | TVAAPSVFIFPPSDEQLKSGTASVVCLLNNFYPREAK | |
| | VQWKVDNALQSGNSQESVTEQDSKDSTYSLSSTLTL | |
| | SKADYEKHKVYACEVTHQGLSSPVTKSFNRGEC | |
| H3L3-VH | QVQLVESGGGVVQPGRSLRLSCLGSGFSFSSYGMN | 13 |
| | WVRQAPGKGLEWVASISPSGSYFYYADSVKGRFTIS | |
| | RDNSKNTLYLQMTSLRAEDTAIYYCARLPGFNPPFD | |
| | HWGQGTLVTVSS | |
| H3L3-VL | DIQMTQSPSSLSASVGDRVTITCKASQNVGGNVDWY | 14 |
| | QWKPGKAPKLLIYGASNRYTGVPDRFRGSGSGTDFT | |
| | LTISSLQPEDVATYYCLQYKNNPWTFGGGTKVEIK | |
| H1L1-Heavy | EVQLVESGGGLVKPGGSLRLSCAASGFSFSSYGMN | 21 |
| Chain #2 | WVRQAPGKGLEWVSSISPSGSYFYYADSVKGRFTIS | |
| | RDNAKNSLYLQMNSLRAEDTAVYYCARLPGFNPPF | |
| | DHWGQGTLVTVSSASTKGPSVFPLAPSSKSTSGGTA | |
| | ALGCLVKDYFPEPVTVSWNSGALTSGVHTFPAVLQS | |
| | SGLYSLSSVVTVPSSSLGTQTYICNVNHKPSNTKVDK | |
| | RVEPKSCDKTHTCPPCPAPEFEGGPSVFLFPPKPKDT | |
| | LMISRTPEVTCVVVDVSHEDPEVKFNWYVDGVEVH | |
| | NAKTKPREEQYNSTYRVVSVLTVLHQDWLNGKEYK | |
| | CKVSNKALPASIEKTISKAKGQPREPQVYTLPPSREE | |
| | MTKNQVSLTCLVKGFYPSDIAVEWESNGQPENNYK | |
| | TTPPVLDSDGSFFLYSKLTVDKSRWQQGNVFSCSVM | |
| | HEALHNHYTQKSLSLSPG | |

In some aspects, the anti-Claudin-1 antibody comprises a complementarity determining region (CDR) H1 comprising the amino acid sequence set forth in SEQ ID NO: 5, a CDR H2 comprising the amino acid sequence set forth in SEQ ID NO: 6, and a CDR H3 comprising the amino acid sequence set forth in SEQ ID NO: 7.

In some aspects, the anti-Claudin-1 antibody comprises a complementarity determining region (CDR) L1 comprising the amino acid sequence set forth in SEQ ID NO: 8, a CDR L2 comprising the amino acid sequence “Gly Ala”, and a CDR L3 comprising the amino acid sequence set forth in SEQ ID NO: 10.

In some aspects, the complementarity determining regions (CDRs) disclosed herein are defined according to IMGT®. However, it is appreciated that other methods of defining the CDRs in the art can also be used.

In some aspects, the anti-Claudin-1 antibody comprises a VH comprising the amino acid sequence set forth in SEQ ID NO: 3 or SEQ ID NO: 13.

In some aspects, the anti-Claudin-1 antibody comprises a VH comprising an amino acid sequence at least 85%, 86%, 87%, 88%, 89%, 90%, 91%, 92%, 93%, 94%, 95%, 96%, 97%, 98%, or 99% identical to SEQ ID NO: 3. In some aspects, the anti-Claudin-1 antibody comprises a VH comprising an amino acid sequence at least 85%, 86%, 87%, 88%, 89%, 90%, 91%, 92%, 93%, 94%, 95%, 96%, 97%, 98%, or 99% identical to SEQ ID NO: 13.

In some aspects, the anti-Claudin-1 antibody comprises a VL comprising the amino acid sequence set forth in SEQ ID NO: 4 or SEQ ID NO: 14.

In some aspects, the anti-Claudin-1 antibody comprises a VL comprising an amino acid sequence at least 85%, 86%, 87%, 88%, 89%, 90%, 91%, 92%, 93%, 94%, 95%, 96%, 97%, 98%, or 99% identical to SEQ ID NO: 4. In some aspects, the anti-Claudin-1 antibody comprises a VL comprising an amino acid sequence at least 85%, 86%, 87%, 88%, 89%, 90%, 91%, 92%, 93%, 94%, 95%, 96%, 97%, 98%, or 99% identical to SEQ ID NO: 14.

In some aspects, the anti-Claudin-1 antibody comprises a VH comprising the amino acid sequence set forth in SEQ ID NO: 3; and a VL comprising the amino acid sequence set forth in SEQ ID NO: 4.

In some aspects, the anti-Claudin-1 antibody comprises a VH comprising the amino acid sequence set forth in SEQ ID NO: 13; and a VL comprising the amino acid sequence set forth in SEQ ID NO: 14.

In some aspects, the anti-Claudin-1 antibody comprises a Heavy Chain comprising the amino acid sequence set forth in SEQ ID NO: 1, SEQ ID NO: 11, or SEQ ID NO: 21.

In some aspects, the anti-Claudin-1 antibody comprises a Heavy Chain comprising an amino acid sequence at least 85%, 86%, 87%, 88%, 89%, 90%, 91%, 92%, 93%, 94%, 95%, 96%, 97%, 98%, or 99% identical to SEQ ID NO: 1. In some aspects, the anti-Claudin-1 antibody comprises a Heavy Chain comprising an amino acid sequence at least 85%, 86%, 87%, 88%, 89%, 90%, 91%, 92%, 93%, 94%, 95%, 96%, 97%, 98%, or 99% identical to SEQ ID NO: 11. In some aspects, the anti-Claudin-1 antibody comprises a Heavy Chain comprising an amino acid sequence at least 85%, 86%, 87%, 88%, 89%, 90%, 91%, 92%, 93%, 94%, 95%, 96%, 97%, 98%, or 99% identical to SEQ ID NO: 21.

In some aspects, the anti-Claudin-1 antibody comprises a Light Chain comprising the amino acid sequence set forth in SEQ ID NO: 2 or SEQ ID NO: 12.

In some aspects, the anti-Claudin-1 antibody comprises a Light Chain comprising an amino acid sequence at least 85%, 86%, 87%, 88%, 89%, 90%, 91%, 92%, 93%, 94%, 95%, 96%, 97%, 98%, or 99% identical to SEQ ID NO: 2.

In some aspects, the anti-Claudin-1 antibody comprises a Light Chain comprising an amino acid sequence at least 85%, 86%, 87%, 88%, 89%, 90%, 91%, 92%, 93%, 94%, 95%, 96%, 97%, 98%, or 99% identical to SEQ ID NO: 12.

In some aspects, the anti-Claudin-1 antibody comprises a Heavy Chain comprising the amino acid sequence set forth in SEQ ID NO: 1; and a Light Chain comprising the amino acid sequence set forth in SEQ ID NO: 2.

In some aspects, the anti-Claudin-1 antibody comprises a Heavy Chain comprising the amino acid sequence set forth in SEQ ID NO: 21; and a Light Chain comprising the amino acid sequence set forth in SEQ ID NO: 2.

In some aspects, the anti-Claudin-1 antibody comprises a Heavy Chain comprising the amino acid sequence set forth in SEQ ID NO: 11; and a Light Chain comprising the amino acid sequence set forth in SEQ ID NO: 12.

In some aspects, the anti-Claudin-1 antibody comprises a complementarity determining region (CDR) H1 comprising the amino acid sequence set forth in SEQ ID NO: 5, a CDR H2 comprising the amino acid sequence set forth in SEQ ID NO: 6, and a CDR H3 comprising the amino acid sequence set forth in SEQ ID NO: 7 and a VH comprising the amino acid sequence set forth in SEQ ID NO: 3. In some aspects, the anti-Claudin-1 antibody comprises a complementarity determining region (CDR) H1 comprising the amino acid sequence set forth in SEQ ID NO: 5, a CDR H2 comprising the amino acid sequence set forth in SEQ ID NO: 6, and a CDR H3 comprising the amino acid sequence set forth in SEQ ID NO: 7 and a VL comprising the amino acid sequence set forth in SEQ ID NO: 4. In some aspects, the anti-Claudin-1 antibody comprises a complementarity determining region (CDR) H1 comprising the amino acid sequence set forth in SEQ ID NO: 5, a CDR H2 comprising the amino acid sequence set forth in SEQ ID NO: 6, and a CDR H3 comprising the amino acid sequence set forth in SEQ ID NO: 7 and a VH comprising the amino acid sequence set forth in SEQ ID NO: 3 and a VL comprising the amino acid sequence set forth in SEQ ID NO: 4.

In some aspects, the anti-Claudin-1 antibody comprises a complementarity determining region (CDR) H1 comprising the amino acid sequence set forth in SEQ ID NO: 5, a CDR H2 comprising the amino acid sequence set forth in SEQ ID NO: 6, and a CDR H3 comprising the amino acid sequence set forth in SEQ ID NO: 7 and a VH comprising the amino acid sequence set forth in SEQ ID NO: 13. In some aspects, the anti-Claudin-1 antibody comprises a complementarity determining region (CDR) H1 comprising the amino acid sequence set forth in SEQ ID NO: 5, a CDR H2 comprising the amino acid sequence set forth in SEQ ID NO: 6, and a CDR H3 comprising the amino acid sequence set forth in SEQ ID NO: 7 and a VL comprising the amino acid sequence set forth in SEQ ID NO: 14. In some aspects, the anti-Claudin-1 antibody comprises a complementarity determining region (CDR) H1 comprising the amino acid sequence set forth in SEQ ID NO: 5, a CDR H2 comprising the amino acid sequence set forth in SEQ ID NO: 6, and a CDR H3 comprising the amino acid sequence set forth in SEQ ID NO: 7 and a VH comprising the amino acid sequence set forth in SEQ ID NO: 13 and a VL comprising the amino acid sequence set forth in SEQ ID NO: 14.

In some aspects, the six complementarity determining regions (CDRs) of the anti-Claudin-1 antibody are the same as those in the anti-Claudin-1 monoclonal antibody secreted by a hybridoma cell line deposited at the DSMZ on Jul. 29, 2008 under an Accession Number DSM ACC2938.

In some aspects, the heavy chain variable region (“VH”) and the light chain variable region (“VL”) of the anti-Claudin-1 antibody are the same as those in the anti-Claudin-1 monoclonal antibody secreted by a hybridoma cell line deposited at the DSMZ on Jul. 29, 2008 under an Accession Number DSM ACC2938.

In some aspects, the heavy chain and light chain of the anti-Claudin-1 antibody are the same as those in the anti-Claudin-1 monoclonal antibody secreted by a hybridoma cell line deposited at the DSMZ on Jul. 29, 2008 under an Accession Number DSM ACC2938.

The humanized anti-Claudin-1 antibody may be a full monoclonal antibody having an isotope selected from the group consisting of IgG1, IgG2, IgG3 and IgG4. Alternatively, the humanized anti-Claudin-1 antibody may be a fragment of a monoclonal antibody, for example, a variable fragment (Fv), an antigen-binding fragment (Fab), a divalent antibody fragment (F(ab′)2), a Fab prime fragment (Fab′), a disulfide-stabilized Fv fragment (dsFv), a single-chain variable fragment (scFv), or a tandem single-chain variable fragment (sc(Fv)2). In some embodiments, the humanized anti-Claudin-1 antibody may be a bispecific antibody, for example, a diabody.

Anti-Claudin-1 antibodies (or biologically active variants or fragments thereof) suitable for use according to the present disclosure may be functionally linked (e.g., by chemical coupling, genetic fusion, non-covalent association or otherwise) to one or more other molecular entities. Methods for the preparation of such modified antibodies (or conjugated antibodies) are known in the art (see, for example, “Affinity Techniques. Enzyme Purification: Part B”, Methods in Enzymol., 1974, Vol. 34, Jakoby and Wilneck (Eds.), Academic Press: New York, NY; and Wilchek and Bayer, Anal. Biochem., 1988, 171:1-32). Preferably, molecular entities are attached at positions on the antibody molecule that do not interfere with the binding properties of the resulting conjugate, e.g., positions that do not participate in the specific binding of the antibody to its target.

The antibody molecule and molecular entity may be covalently, directly linked to each other. Or, alternatively, the antibody molecule and molecular entity may be covalently linked to each other through a linker group. This can be accomplished by using any of a wide variety of stable bifunctional agents well known in the art, including homofunctional and heterofunctional linkers.

In some aspects, an anti-Claudin-1 antibody (or a biologically active fragment thereof) for use according to the present disclosure is conjugated to a detectable agent. Any of a wide variety of detectable agents can be used, including, without limitation, various ligands, radionuclides (e.g., 3H, 125I, 131I, and the like), fluorescent dyes (e.g., fluorescein isothiocyanate, rhodamine, phycoerytherin, phycocyanin, allophycocyanin, o-phthalaldehyde and fluorescamine), chemiluminescent agents (e.g., luciferin, luciferase and aequorin), microparticles (such as, for example, quantum dots, nanocrystals, phosphors and the like), enzymes (such as, for example, those used in an ELISA, i.e., horseradish peroxidase, beta-galactosidase, luciferase, alkaline phosphatase), colorimetric labels, magnetic labels, and biotin, dioxigenin or other haptens and proteins for which antisera or monoclonal antibodies are available.

Other molecular entities that can be conjugated to an anti-Claudin-1 antibody of the present disclosure (or a biologically active fragment thereof) include, but are not limited to, linear or branched hydrophilic polymeric groups, fatty acid groups, or fatty ester groups.

Thus, in the practice of the present disclosure, anti-Claudin-1 antibodies can be used in the form of full length antibodies, biologically active variants or fragments thereof, chimeric antibodies, humanized antibodies, and antibody-derived molecules comprising at least one complementarity determining region (CDR) from either a heavy chain or light chain variable region of an anti-Claudin-1 antibody, including molecules such as Fab fragments, F(ab′)2 fragments, Fd fragments, Fabc fragments, Sc antibodies (single chain antibodies), diabodies, individual antibody light single chains, nanobodies, individual antibody heavy chains, heavy chain only antibodies, chimeric fusions between antibody chains and other molecules, and antibody conjugates, such as antibodies conjugated to a therapeutic agent or a detectable agent. Preferably, anti-Claudin-1 antibody-related molecules according to the present disclosure retain the antibody's ability to bind its antigen, in particular the extracellular domain of Claudin-1.

### Chimeric Antigen Receptors

Chimeric antigen receptor (CAR) T-cell therapy, or CAR T-cell therapy, is a cancer treatment, based on the use of T cells genetically engineered to express a synthetic receptor that binds a tumor antigen. Engineered CAR T cells are expanded in vitro and infused into the patient's body to attack and destroy chemotherapy-resistant cancer.

The term “chimeric antigen receptor” (CAR) refers to molecules that combine a binding domain against a component present on the target cell, for example an antibody-based specificity for a desired antigen (e.g., a tumor antigen, such as CLDN1) with a T cell receptor-activating intracellular domain to generate a chimeric protein that exhibits a specific anti-target cellular immune activity.

A “signal transducing domain” or “signaling domain” of a CAR is responsible for intracellular signaling following the binding of an extracellular ligand binding domain to the target resulting in the activation of the immune cell and immune response. In other words, the signal transducing domain is responsible for the activation of at least one of the normal effector functions of the immune cell in which the CAR is expressed. For example, the effector function of a T cell can be a cytolytic activity or helper activity including the secretion of cytokines. Thus, the term “signal transducing domain” refers to the portion of a protein which transduces the effector function signal and directs the cell to perform a specialized function. Examples of signal transducing domains for use in a CAR include the cytoplasmic sequences of the T cell receptor and co-receptors that act in concert to initiate signal transduction following antigen receptor engagement, as well as any derivate or variant of these sequences and any synthetic sequence that has the same functional capability. In some cases, signaling domains comprise two distinct classes of cytoplasmic signaling sequences, those that initiate antigen-dependent primary activation, and those that act in an antigen-independent manner to provide a secondary or co-stimulatory signal. Primary cytoplasmic signaling sequences can comprise signaling motifs which are known as immunoreceptor tyrosine-based activation motifs of ITAMs. ITAMs are well defined signaling motifs found in the intracytoplasmic tail of a variety of receptors that serve as binding sites for syk/zap70 class tyrosine kinases. Exemplary ITAMs include those derived from TCRzeta, FcRgamma, FcRbeta, FcRepsilon, CD3gamma, CD3delta, CD3epsilon, CD5, CD22, CD79a, CD79b and CD66d. In some aspects, the signal transducing domain of the CAR can comprise the CD3zeta signaling domain (SEQ ID NO: 15).

Generally, CARs are synthetic receptors consisting of a targeting moiety that is associated with one or more signaling domains in a single fusion molecule. In general, the binding moiety of a CAR consists of an antigen-binding domain of a single-chain antibody (scFv), comprising the light and heavy chain variable fragments of a monoclonal antibody joined by a flexible linker. This molecule is generally joined to an intracellular signaling molecule, comprising one or more intracellular signaling domains that mediate T-cell activation. The signaling domains for first generation CARs are generally derived from the cytoplasmic region of the CD3zeta or the Fc receptor gamma chains (or the intracellular signaling domain of another immune-receptor-tyrosine-based-activation-motif [ITAM]-containing protein). First generation CARs have been shown to successfully redirect T-cell cytotoxicity. However, they failed to provide prolonged expansion and anti-tumor activity in vivo. Signaling domains from co-stimulatory molecules, as well as transmembrane and hinge domains have been added to form CARs of second, third, and fourth generations. Second generation chimeric receptors generally incorporate a costimulatory endodomain (e.g., 4-1BB/CD3ζ). Third generation CARs generally contain multiple costimulatory signaling modules. Fourth-generation CARs were generated by adding IL-12 to the base of the second-generation constructs, and are known as T cell redirected for universal cytokine-mediated killing (TRUCKs). TRUCKs augment T-cell activation and activate and attract innate immune cells to eliminate antigen-negative cancer cells in the targeted lesion. Therapeutic trials in humans using CAR T-cell therapy have shown some success. For example, CAR redirected T cells specific for the B cell differentiation antigen CD19 have shown dramatic efficacy in the treatment of B cell malignancies, while TCR-redirected T cells have shown benefits in patients suffering from solid cancer. Stauss et al. describe strategies to modify therapeutic CARs and TCRs, for use in the treatment of cancer, for example, to enhance the antigen-specific effector function and limit toxicity of engineered T cells (Current Opinion in Pharmacology 2015, 24:113-118).

In some aspects, provided herein is a CAR which is specific for Claudin-1, which is expressed on the surface of cancer cells. In some aspects of the present disclosure, the CAR comprises an extracellular target-specific binding domain, a transmembrane domain, an intracellular signaling domain (such as a signaling domain derived from CD3zeta or FcRgamma), and/or one or more co-stimulatory signaling domains derived from a co-stimulatory molecule, such as, but not limited to, 4-1BB. In some aspects, the CAR includes a hinge or spacer region between the extracellular binding domain and the transmembrane domain, such as a CD8alpha hinge. In some aspects, the CAR comprises an extracellular target-specific binding domain, which is an anti-Claudin single chain antibody (scFv), and may be a murine, human or humanized scFv. Single chain antibodies may be cloned from the V region genes of a hybridoma specific for a desired target. A technique which can be used for cloning the variable region heavy chain (VH) and variable region light chain (VL) has been described, for example, in Orlandi et al., PNAS 86:3833-3837 (1989). Thus, in some aspects, a binding domain comprises an antibody-derived binding domain but can be a non-antibody derived binding domain. An antibody-derived binding domain can be a fragment of an antibody or a genetically engineered product of one or more fragments of the antibody, which fragment is involved in binding with the antigen.

In some aspects, the CARs of the present disclosure may comprise a linker between the various domains, added for appropriate spacing and conformation of the molecule. For example, in some aspects, there may be a linker between the binding domain VH or VL which may be between 1-10 amino acids long. In some aspects, the linker between any of the domains of the chimeric antigen receptor may be between 1-20 or 20 amino acids long. In this regard, the linker may be 1, 2, 3, 4, 5, 6, 7, 8, 9, 10, 11, 12, 13, 14, 15, 16, 17, 18, 19 or 20 amino acids long. In some aspects, the linker may be 21, 22, 23, 24, 25, 26, 27, 28, 29 or 30 amino acids long. Ranges including the numbers described herein are also included herein, e.g., a linker 10-30 amino acids long.

In some aspects, linkers suitable for use in the CAR described herein are flexible linkers. Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Exemplary flexible linkers include glycine polymers (G) n, glycine-serine polymers, where n is an integer of at least one, glycine-alanine polymers, alanine-serine polymers, and other flexible linkers known in the art. Glycine and glycine-serine polymers are relatively unstructured, and therefore may be able to serve as a neutral tether between domains of fusion proteins such as the CARs described herein. Glycine accesses significantly more phipsi space than even alanine, and is much less restricted than residues with longer side chains (see Scheraga, Rev. Computational Chem. 11173-142 (1992)). The ordinarily skilled artisan will recognize that design of a CAR can include linkers that are all or partially flexible, such that the linker can include a flexible linker as well as one or more portions that confer less flexible structure to provide for a desired CAR structure. Specific linkers include (G4S)n linkers, wherein n=1-3. In some aspects, the linker comprises the amino acid sequence of SEQ ID NO: 16 or SEQ ID NO: 17.

The binding domain of the CAR may be followed by a “spacer,” or, “hinge,” which refers to the region that moves the antigen binding domain away from the effector cell surface to enable proper cell/cell contact, antigen binding and activation (Patel et al., Gene Therapy 6:412-419 (1999)). The hinge region in a CAR is generally between the transmembrane (TM) and the binding domain. In some aspects, a hinge region is an immunoglobulin hinge region and may be a wild type immunoglobulin hinge region or an altered wild type immunoglobulin hinge region. Other exemplary hinge regions used in the CARs described herein include the hinge region derived from the extracellular regions of type 1 membrane proteins such as CD8alpha, CD4, CD28 and CD7, which may be wild-type hinge regions from these molecules or may be altered. In some aspects, the hinge region comprises a CD8alpha hinge (SEQ ID NO: 18).

The “transmembrane” region or domain is the portion of the CAR that anchors the extracellular binding portion to the plasma membrane of the immune effector cell, and facilitates binding of the binding domain to the target antigen. The transmembrane domain may be a CD3zeta transmembrane domain, however other transmembrane domains that may be employed include those obtained from CD8alpha, CD4, CD28, CD45, CD9, CD16, CD22, CD33, CD64, CD80, CD86, CD134, CD137, and CD154. In some aspects, the transmembrane domain is the transmembrane domain of CD137. In some aspects, the transmembrane domain comprises the amino acid sequence of SEQ ID NO: 19. In some aspects, the transmembrane domain is synthetic, in which case it would comprise predominantly hydrophobic residues such as leucine and valine.

As described above, the “intracellular signaling domain” or “signaling domain” refers to the part of the chimeric antigen receptor protein that participates in transducing the message of effective CAR binding to a target antigen into the interior of the immune effector cell to elicit effector cell function, e.g., activation, cytokine production, proliferation and cytotoxic activity, including the release of cytotoxic factors to the CAR-bound target cell, or other cellular responses elicited with antigen binding to the extracellular CAR domain. The term “effector function” refers to a specialized function of the cell. Effector function of the T cell, for example, may be cytolytic activity or help or activity including the secretion of a cytokine. Thus, the terms “intracellular signaling domain” or “signaling domain,” used interchangeably herein, refer to the portion of a protein which transduces the effector function signal and that directs the cell to perform a specialized function. While usually the entire intracellular signaling domain can be employed, in many cases it is not necessary to use the entire domain. To the extent that a truncated portion of an intracellular signaling domain is used, such truncated portion may be used in place of the entire domain as long as it transduces the effector function signal. The term intracellular signaling domain is meant to include any truncated portion of the intracellular signaling domain sufficient to transducing effector function signal. The intracellular signaling domain is also known as the “signal transduction domain,” and is typically derived from portions of the human CD3 or FcRγ chains.

It is known in the art that signals generated through the T cell receptor alone are insufficient for full activation of the T cell and that a secondary, or costimulatory signal is also required. Thus, T cell activation can be said to be mediated by two distinct classes of cytoplasmic signaling sequences: those that initiate antigen dependent primary activation through the T cell receptor (primary cytoplasmic signaling sequences) and those that act in an antigen independent manner to provide a secondary or costimulatory signal (secondary cytoplasmic signaling sequences). Cytoplasmic signaling sequences that act in a costimulatory manner may contain signaling motifs which are known as immunoreceptor tyrosine-based activation motif or ITAMs.

Examples of ITAM containing primary cytoplasmic signaling sequences that are of particular use in the CARs disclosed herein include those derived from TCRzeta, FcRgamma, FcRbeta, CD3gamma, CD3delta, CD3epsilon, CD5, CD22, CD79a, CD79b and CD66d. In one some aspects, the intracellular signaling domain of the anti-BCMA CARs described herein are derived from CD3zeta. In some aspects, the signaling domain comprises the amino acid sequence of SEQ ID NO: 15.

As used herein, the term, “costimulatory signaling domain,” or “costimulatory domain”, refers to the portion of the CAR comprising the intracellular domain of a costimulatory molecule. Costimulatory molecules are cell surface molecules other than antigen receptors or Fc receptors that provide a second signal required for efficient activation and function of T lymphocytes upon binding to antigen. Examples of such co-stimulatory molecules include CD27, CD28, 4-1BB (CD137), OX40 (CD134), CD30, CD40, PD-1, ICOS (CD278), LFA-1, CD2, CD7, LIGHT, NKD2C, B7-H2 and a ligand that specifically binds CD83. Accordingly, while the present disclosure provides exemplary sequences for costimulatory domains derived from CD3zeta and 4-1BB, other costimulatory domains are contemplated for use with the CARs described herein. The inclusion of one or more co-stimulatory signaling domains may enhance the efficacy and expansion of T cells expressing CAR receptors. The intracellular signaling and costimulatory signaling domains may be linked in any order in tandem to the carboxyl terminus of the transmembrane domain. In some aspects, the costimulatory domain comprises the amino acid sequence of SEQ ID NO: 20.

In some aspects, the anti-Claudin-1 CARs of the present disclosure comprise any of the elements of Table 2.

TABLE 2
CAR elements

| | | SEQ |
|---|---|---|
| Description | Sequence | ID NO: |
| CD3zeta | RVKFSRSADAPAYKQGQNQLYNELNLGRREEYDVL | 15 |
| signaling | DKRRGRDPEMGGKPRRKNPQEGLYNELQKDKMAEA | |
| domain | YSEIGMKGERRRGKGHDGLYQGLSTATKDTYDALH | |
| | MQALPPR | |
| (G4S) | GGGGSGGGGSGGGGS | 16 |
| linker 1 | | |
| linker 2 | GSTSGSGKPGSGEGSTKG | 17 |
| CD8alpha | TTTPAPRPPTPAPTIASQPLSLRPEACRPAAGGAVHTR | 18 |
| hinge | GLDFACD | |
| TM domain | IYIWAPLAGTCGVLLLSLVITLYC | 19 |
| costimulatory | KRGRKKLLYIFKQPFMRPVQTTQEEDGCSCRFPEEEE | 20 |
| domain | GGCEL | |

Although scFv-based CARs engineered to contain a signaling domain from CD3 or FcRgamma have been shown to deliver a potent signal for T cell activation and effector function, they are usually not sufficient to elicit signals that promote T cell survival and expansion in the absence of a concomitant costimulatory signal. Other CARs containing a binding domain, a hinge, a transmembrane and the signaling domain derived from CD3zeta or FcRgamma together with one or more costimulatory signaling domains (e.g., intracellular costimulatory domains derived from CD28, CD137, CD134 and CD278) may more effectively direct antitumor activity as well as increased cytokine secretion, lytic activity, survival and proliferation in CAR expressing T cells in vitro, and in animal models and cancer patients (Milone et al., Molecular Therapy 17:1453-1464 (2009); Zhong et al., Molecular Therapy 18:413-420 (2010); Carpenito et al., PNAS 106:3360-3365 (2009)).

In some aspects, the anti-Claudin-1 CAR of the disclosure comprises (a) an anti-Claudin-1 binding domain (e.g., an scFv having binding regions (e.g., CDRs or variable domains) from any one or more of the sequences identified in Table 1), (b) a hinge region derived from human CD8alpha, (c) a human CD8alpha transmembrane domain, and (d) a human T cell receptor CD3 zeta chain (CD3) intracellular signaling domain, and optionally one or more costimulatory signaling domains, e.g., 4-1BB.

The anti-Claudin-1 binding domain may comprise any of the VH and VL sequences set forth herein, for example the anti-Claudin-1 binding domain may comprise a VH comprising the sequence set forth in SEQ ID NOs. 3 or 13 and a VL comprising the sequence set forth in SEQ ID NOs. 4 or 14. In some embodiments, the anti-Claudin-1 binding domain comprises a CDR H1 comprising the amino acid sequence set forth in SEQ ID NO: 5, a CDR H2 comprising the amino acid sequence set forth in SEQ ID NO: 6, and a CDR H3 comprising the amino acid sequence set forth in SEQ ID NO: 7. In some embodiments, the anti-Claudin-1 binding domain comprises a complementarity determining region (CDR) L1 comprising the amino acid sequence set forth in SEQ ID NO: 8, a CDR L2 comprising the amino acid sequence “Gly Ala”, and a CDR L3 comprising the amino acid sequence set forth in SEQ ID NO: 10. In some embodiments, the anti-Claudin-1 binding domain comprises a VH comprising an amino acid sequence at least 85%, 86%, 87%, 88%, 89%, 90%, 91%, 92%, 93%, 94%, 95%, 96%, 97%, 98%, or 99% identical to SEQ ID NO: 3. In some aspects, In some embodiments, the anti-Claudin-1 binding domain comprises a VH comprising an amino acid sequence at least 85%, 86%, 87%, 88%, 89%, 90%, 91%, 92%, 93%, 94%, 95%, 96%, 97%, 98%, or 99% identical to SEQ ID NO: 13. In some embodiments, the anti-Claudin-1 binding domain comprises a VL comprising an amino acid sequence at least 85%, 86%, 87%, 88%, 89%, 90%, 91%, 92%, 93%, 94%, 95%, 96%, 97%, 98%, or 99% identical to SEQ ID NO: 4. In some embodiments, the anti-Claudin-1 binding domain comprises a VL comprising an amino acid sequence at least 85%, 86%, 87%, 88%, 89%, 90%, 91%, 92%, 93%, 94%, 95%, 96%, 97%, 98%, or 99% identical to SEQ ID NO: 14. In some embodiments, the anti-Claudin-1 binding domain comprises a VH comprising the amino acid sequence set forth in SEQ ID NO: 3; and a VL comprising the amino acid sequence set forth in SEQ ID NO: 4. In some embodiments, the anti-Claudin-1 binding domain comprises a VH comprising the amino acid sequence set forth in SEQ ID NO: 13; and a VL comprising the amino acid sequence set forth in SEQ ID NO: 14.

In some aspects, the different protein domains are arranged from amino to carboxyl terminus in the following order from N-terminus to C-terminus: an anti-Claudin-1 binding domain, a hinge region and a transmembrane domain. The intracellular signaling domain and optional co-stimulatory signaling domains are linked to the transmembrane carboxy terminus in any order in tandem to form a single chain chimeric polypeptide.

### Polynucleotides and Vectors

In some aspects, provided herein are polynucleotides encoding the CARs described herein. In some embodiments, a nucleic acid construct encoding an anti-Claudin-1 CAR is a chimeric nucleic acid molecule comprising a nucleic acid molecule comprising different coding sequences, for example, (5′ to 3′) the coding sequences of an anti-Claudin-1 scFv, a human CD8alpha-hinge, a human CD8alpha transmembrane domain and a CD3zeta intracellular signaling domain. In some aspects, a nucleic acid construct encoding an anti-Claudin-1 CAR is a chimeric nucleic acid molecule comprising a nucleic acid molecule comprising different coding sequences, for example, (5′ to 3′) the coding sequences of an anti-Claudin-1 scFv, a human CD8alpha-hinge, a human CD8alpha transmembrane domain, a 4-1BB co-stimulatory domain, and a CD3zeta co-stimulatory domain.

In some aspects, the polynucleotide encoding the CAR described herein is inserted into a vector. For expression of an anti-Claudin-1 CAR, the vector may be introduced into a host cell to allow expression of the polypeptide within the host cell. The expression vectors may contain a variety of elements for controlling expression, including without limitation, promoter sequences, transcription initiation sequences, enhancer sequences, selectable markers, and signal sequences. These elements may be selected as appropriate by a person of ordinary skill in the art, as described above. For example, the promoter sequences may be selected to promote the transcription of the polynucleotide in the vector. Suitable promoter sequences include, without limitation, T7 promoter, T3 promoter, SP6 promoter, beta-actin promoter, EF1a promoter, CMV promoter, and SV40 promoter. Enhancer sequences may be selected to enhance the transcription of the polynucleotide. Selectable markers may be selected to allow selection of the host cells inserted with the vector from those not, for example, the selectable markers may be genes that confer antibiotic resistance. Signal sequences may be selected to allow the expressed polypeptide to be transported outside of the host cell.

In some embodiments, the vector is a plasmid, for example, plasmid DNA, phage DNA, a bacterial plasmid, or phage DNA. In some embodiments, the vector is a viral vector, for example, a retrovirus, an adenovirus, a vaccinia virus, or a baculovirus.

### Modified Cells

The CARs of the present disclosure may be introduced into a host cell using transfection and/or transduction techniques known in the art. As used herein, the terms, “transfection,” and, “transduction,” refer to the processes by which an exogenous nucleic acid sequence is introduced into a host cell. The nucleic acid may be integrated into the host cell DNA or may be maintained extrachromosomally. The nucleic acid may be maintained transiently or may be a stable introduction. Transfection may be accomplished by a variety of means known in the art including, but not limited to, calcium phosphate-DNA co-precipitation, DEAE-dextranmediated transfection, polybrene-mediated transfection, electroporation, microinjection, liposome fusion, lipofection, protoplast fusion, retroviral infection, and biolistics. Transduction refers to the delivery of a gene(s) using a viral or retroviral vector by means of viral infection rather than by transfection.

As used herein, the term “genetically engineered” or “genetically modified” in the context of a cell refers to the addition of extra genetic material in the form of DNA or RNA into the total genetic material in a cell. The terms, “genetically modified cells,” “modified cells,” and, “redirected cells,” are used interchangeably.

In some aspects, the CAR of the present disclosure is introduced and expressed in immune effector cells so as to redirect their specificity to a target antigen of interest, e.g. Claudin-1.

The present disclosure provides methods for making the immune effector cells which express the CAR as described herein. In some aspects, the method comprises transfecting or transducing immune effector cells isolated from a subject, such as a subject having a Claudin-1 expressing tumor cell, such that the immune effector cells express one or more CAR as described herein. In some aspects, the immune effector cells are isolated from an individual and genetically modified without further manipulation in vitro. Such cells can then be directly re-administered into the individual. In some aspects, the immune effector cells are first activated and stimulated to proliferate in vitro prior to being genetically modified to express a CAR. In this regard, the immune effector cells may be cultured before or after being genetically modified (i.e., transduced or transfected to express a CAR as described herein).

Prior to in vitro manipulation or genetic modification of the immune effector cells described herein, the source cells may be obtained from a subject. In some aspects, the immune effector cells for use with the CARs as described herein comprise T cells. T cells can be obtained from a number of sources, including peripheral blood mononuclear cells, bone marrow, lymph nodes tissue, cord blood, thymus issue, tissue from a site of infection, ascites, pleural effusion, spleen tissue, and tumors. The cells may be autologous or allogeneic. In some aspects, T cell can be obtained from a unit of blood collected from the subject using any number of techniques known to the skilled person, such as FICOLL separation. In some aspects, cells from the circulating blood of an individual are obtained by apheresis. The apheresis product typically contains lymphocytes, including T cells, monocytes, granulocyte, B cells, other nucleated white blood cells, red blood cells, and platelets. In some aspects, the cells collected by apheresis may be washed to remove the plasma fraction and to place the cells in an appropriate buffer or media for subsequent processing. In some aspects, the cells are washed with PBS. In some aspects, the washed solution lacks calcium, and may lack magnesium or may lack many, if not all, divalent cations. As would be appreciated by those of ordinary skill in the art, a washing step may be accomplished by methods known to those in the art, such as by using a semiautomated flow through centrifuge. After washing, the cells may be resuspended in a variety of biocompatible buffers or other saline solution with or without buffer. In some aspects, the undesirable components of the apheresis sample may be removed in the cell directly resuspended culture media.

In some aspects, T cells are isolated from peripheral blood mononuclear cells (PBMCs) by lysing the red blood cells and depleting the monocytes, for example, by centrifugation through a PERCOLL™ gradient. A specific subpopulation of T cells, such as CD28+, CD4+, CD8+, CD45RA+, and CD45RO+ T cells, can be further isolated by positive or negative selection techniques. For example, enrichment of a T cell population by negative selection can be accomplished with a combination of antibodies directed to surface markers unique to the negatively selected cells. One method for use herein is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD1b, CD16, HLA-DR, and CD8. Flow cytometry and cell sorting may also be used to isolate cell populations of interest for use according to the present disclosure.

PBMCs may be used directly for genetic modification with the CARs using methods as described herein. In some aspects, after isolation of PBMC, T lymphocytes are further isolated and in some aspects, both cytotoxic and helper T lymphocytes can be sorted into naive, memory, and effector T cell subpopulations either before or after genetic modification and/or expansion. CD8+ cells can be obtained by using standard methods. In some aspects, CD8+ cells are further sorted into naive, central memory, and effector cells by identifying cell surface antigens that are associated with each of those types of CD8+ cells. In some aspects, memory T cells are present in both CD62L+ and CD62L-subsets of CD8+ peripheral blood lymphocytes. PBMC are sorted into CD62L-CD8+ and CD62L+CD8+ fractions after staining with anti-CD8 and anti-CD62L antibodies. In some aspects, the expression of phenotypic markers of central memory TCM include CD45RO, CD62L, CCR7, CD28, CD3, and CD127 and are negative for granzyme B. In some aspects, central memory T cells are CD45RO+, CD62L+, CD8+ T cells. In some aspects, effector T cells are negative for CD62L, CCR7, CD28, and CD127, and positive for granzyme B and perforin. In some aspects, naive CD8+ T lymphocytes are characterized by the expression of phenotypic markers of naive T cells including CD62L, CCR7, CD28, CD3, CD 127, and CD45RA.

In some aspects, CD4+ T cells are further sorted into subpopulations. For example, CD4+ T helper cells can be sorted into naive, central memory, and effector cells by identifying cell populations that have cell surface antigens. CD4+ lymphocytes can be obtained by standard methods. In some aspects, naive CD4+ T lymphocytes are CD45RO−, CD45RA+, CD62L+CD4+ T cell. In some aspects, central memory CD4+ cells are CD62L positive and CD45RO positive. In some aspects, effector CD4+ cells are CD62L and CD45RO negative.

The immune effector cells, such as T cells, can be genetically modified following isolation using known methods, or the immune effector cells can be activated and expanded (or differentiated in the case of progenitors) in vitro prior to being genetically modified. In some aspects, the immune effector cells, such as T cells, are genetically modified with the chimeric antigen receptors described herein (e.g., transduced with a viral vector comprising a nucleic acid encoding a CAR) and then are activated and expanded in vitro. Methods for activating and expanding T cells are known in the art and are described, for example, in U.S. Pat. Nos. 6,905,874; 6,867,041; 6,797,514; WO2012079000. Generally, such methods include contacting PBMC or isolated T cells with a stimulatory agent and costimulatory agent, such as anti-CD3 and anti-CD28 antibodies, generally attached to a bead or other surface, in a culture medium with appropriate cytokines, such as IL-2. Anti-CD3 and anti-CD28 antibodies attached to the same bead serve as a “surrogate” antigen presenting cell (APC). In some aspects, the T cells may be activated and stimulated to proliferate with feeder cells and appropriate antibodies and cytokines using methods such as those described in U.S. Pat. Nos. 6,040,177; 5,827,642; and WO2012129514.

### Biomarkers

Provided herein are biomarkers that may be used to identify patients suitable for treatment with anti-Claudin-1 antibodies and to guide treatment strategy. In some embodiments, the biomarker is PRO-C3. In some embodiments, the biomarker is C4G. In some embodiments, the biomarker is a combination of PRO-C3 and C4G.

### PRO-C3

Together with type I collagen, type III collagen constitutes the major structural proteins in the human body, in which type III collagen is crucial for type I collagen fibrillogenesis except in bones, which almost exclusively consist of type I collagen (Bao, X., et al., Journal of genetics and genomics 34 (3): 223-228 (2014); Jensen, L T, and Høst, NB, Cardiovascular research 33 (3): 535-539 (1997)). During fibrillar assembly the N-terminal propeptide of type III collagen is cleaved off by specific N-proteases prior to incorporation of the mature collagen in the extracellular matrix (ECM), thus released in the ECM and into circulation. The propeptide molecule consists of three identical a-chains with a total molecular weight of 42 kDa. The removal of the propeptide is sometimes incomplete leaving the propeptide attached to the molecule resulting in thin fibrils with abnormally cross-links and thereby prone to rapid metabolic turnover (Niemelä, O., et al., The Biochemical journal 232 (1): 145-150 (1985); Wang, W., et al., The Biochemical journal 398 (3): 515-519 (2006)), thus PIIINP can both be a marker of formation and degradation. Enzyme-linked immunosorbent assay (ELISA) towards the N-terminal propeptide of type III collagen (“PRO-C3”) have been developed to assess true formation by development of monoclonal antibody towards the N-protease generated neo-epitope of N-terminal propeptide of type III collagen (Nielsen, M., et al., American journal of translational research 5 (3): 303-315 (19 Apr. 2013)).

In some aspects, PRO-C3 can be used as a biomarker to measure anti-CLDN1-antibody target engagement in a cancer.

In some aspects, the cancer is selected from the group consisting of a head and neck cancer (e.g., Head and Neck Squamous Cell Carcinoma), a lung cancer, a breast cancer, a melanoma, a colorectal cancer, a pancreatic cancer, an esophageal cancer, a cholangiocarcinoma, and a hepatocellular carcinoma.

In some aspects, PRO-C3 levels are measured by an enzyme-linked immunoassay (ELISA). In some aspects, PRO-C3 levels are measured by mass spectrometry.

### C4G

Granzyme B degraded type IV collagen products (C4G) is a biomarker measuring granzyme B degraded type IV collagen products (C4G) in serum (Jensen, C., et al., Cancers 12 (10): 2786 (2020)). The discovery of C4G was based on a study from Prakash et al. showing that granzyme B promotes cytotoxic lymphocyte transmigration over the basement membrane by degrading components such as type IV collagen (Prakash, M., et al., Immunity 41 (6): 960-972 (2014)).

In some aspects, C4G levels are measured by an enzyme-linked immunoassay (ELISA). In some aspects, C4G levels are measured by mass spectrometry.

### Detection Methods

In certain aspects, identifying a patient suitable for an anti-Claudin-1 antibody therapy for the present methods includes measuring or assessing a PRO-C3 and/or C4G expression in a sample, for example, a cancer test tissue sample. The methods of measuring or assessing the PRO-C3 and/or C4G expression can be achieved by any methods applicable.

In order to assess the PRO-C3 and/or C4G expression, a test tissue sample may be obtained from the patient who is in need of the therapy. A test tissue sample includes, but is not limited to, any clinically relevant tissue sample, such as a tumor biopsy, a core biopsy tissue sample, a fine needle aspirate, or a sample of bodily fluid, such as blood, plasma, serum, lymph, ascites fluid, cystic fluid, or urine. In some aspects, the test tissue sample is from a primary tumor. In some aspects, the test tissue sample is from a metastasis. In some aspects, test tissue samples are taken from a subject at multiple time points, for example, before treatment, during treatment, and/or after treatment. In some aspects, test tissue samples are taken from different locations in the subject, for example, a sample from a primary tumor and a sample from a metastasis in a distant location. In some aspects, the test tissue sample is from a cancer tissue sample.

In some aspects, the test tissue sample is a paraffin-embedded fixed tissue sample. In some aspects, the test tissue sample is a formalin-fixed paraffin embedded (FFPE) tissue sample. In some aspects, the test tissue sample is a fresh tissue (e.g., tumor) sample. In some aspects, the test tissue sample is a frozen tissue sample. In some aspects, the test tissue sample is a fresh frozen (FF) tissue (e.g., tumor) sample. In some aspects, the test tissue sample is a cell isolated from a fluid. In some aspects, the test tissue sample comprises circulating tumor cells (CTCs). In some aspects, the test tissue sample comprises tumor-infiltrating lymphocytes (TILs). In some aspects, the test tissue sample comprises tumor cells and tumor-infiltrating lymphocytes (TILs). In some aspects, the test tissue sample comprises circulating lymphocytes. In some aspects, the test tissue sample is an archival tissue sample. In some aspects, the test tissue sample is an archival tissue sample with known diagnosis, treatment, and/or outcome history. In some aspects, the sample is a block of tissue. In some aspects, the test tissue sample is dispersed cells. In some aspects, the sample size is from about 1 cell to about 1×106 cells or more. In some aspects, the sample size is about 1 cell to about 1×105 cells. In some aspects, the sample size is about 1 cell to about 10,000 cells. In some aspects, the sample size is about 1 cell to about 1,000 cells. In some aspects, the sample size is about 1 cells to about 100 cells. In some aspects, the sample size is about 1 cell to about 10 cells. In some aspects, the sample size is a single cell.

In some aspects, the test tissue sample is obtained from a liquid biopsy. In some aspects, the test tissue sample is urine, plasma, or serum. In some aspects, the test tissue sample is obtained by a nasal brush. In some aspects, the test tissue sample is obtained by a hair biopsy or scalp biopsy. In some aspects, the test tissue sample is obtained by bronchoalveolar lavage.

In some aspects, the assessment of PRO-C3 and/or C4G expression can be achieved without obtaining a test tissue sample.

In some aspects, selecting a suitable patient includes (i) optionally providing a test tissue sample obtained from a patient with cancer of the tissue, the test tissue sample comprising cancer cells; and (ii) assessing the proportion of cells in the test tissue sample that express PRO-C3 and/or C4G on the surface of the cells based on an assessment that the proportion of cells in the test tissue sample that express PRO-C3 and/or C4G on the cell surface is higher than a predetermined threshold level.

In any of the methods comprising the measurement of PRO-C3 and/or C4G expression in a test tissue sample, however, it should be understood that the step comprising the provision of a test tissue sample obtained from a patient is an optional step. That is, in some aspects, the method includes this step, and in other aspects, this step is not included in the method. It should also be understood that in certain aspects the “measuring” or “assessing” step to identify, or determine the number or proportion of, cells in the test tissue sample that express PRO-C3 and/or C4G is performed by a transformative method of assaying for PRO-C3 and/or C4G expression, for example by performing an IHC assay. In some other aspects, no transformative step is involved and PRO-C3 and/or C4G expression is assessed by, for example, reviewing a report of test results from a laboratory. In some aspects, PRO-C3 and/or C4G expression is assessed by reviewing the results of an immunohistochemistry assay from a laboratory. In some aspects, the steps of the methods up to, and including, assessing PRO-C3 and/or C4G expression provides an intermediate result that may be provided to a physician or other healthcare provider for use in selecting a suitable candidate for an anti-Claudin-1 antibody therapy. In some aspects, the steps of the methods up to, and including, assessing PRO-C3 and/or C4G expression provides an intermediate result that may be provided to a physician or other healthcare provider for use in selecting a suitable candidate for treatment. In certain aspects, the steps of the methods up to, and including, assessing PRO-C3 and/or C4G expression provides an intermediate result that may be provided to a physician or other healthcare provider for use in selecting a suitable candidate for an anti-Claudin-1 antibody therapy. In some aspects, the steps that provide the intermediate result is performed by a medical practitioner or someone acting under the direction of a medical practitioner. In other aspects, these steps are performed by an independent laboratory or by an independent person such as a laboratory technician.

In other aspects, the amount of PRO-C3 and/or C4G is assessed by performing an assay to detect the presence of PRO-C3 and/or C4G polypeptide. In further aspects, the presence of PRO-C3 and/or C4G polypeptide is detected by IHC, enzyme-linked immunosorbent assay (ELISA), in vivo imaging, or flow cytometry. In some aspects, PRO-C3 and/or C4G expression is assayed by IHC. In other aspects of all of these methods, cell surface expression of PRO-C3 and/or C4G is assayed using, e.g., IHC or in vivo imaging.

In some aspects of any of the present methods, the proportion of cells that express PRO-C3 and/or C4G in the test tissue sample is assessed by performing an assay to detect the presence of PRO-C3 and/or C4G polypeptide. In some aspects, the presence of PRO-C3 and/or C4G polypeptide is detected by an immunohistochemistry assay. In some aspects, the test tissue sample is a tumor biopsy. In some aspects, the test tissue sample is a formalin-fixed paraffin embedded (FFPE) sample.

In some aspects, the immunohistochemistry assay is a monoplex assay. In some aspects, the immunohistochemistry assay is a multiplex assay.

In some aspects of the present methods, an automated IHC method is used to assay the expression of PRO-C3 and/or C4G in FFPE tissue specimens. This disclosure provides methods for detecting the presence of human PRO-C3 and/or C4G antigen in a test tissue sample, or quantifying the level of human PRO-C3 and/or C4G antigen or the proportion of cells in the sample that express the antigen, which methods comprise contacting the test sample, and a negative control sample, with an antibody that specifically binds to human PRO-C3 and/or C4G, under conditions that allow for formation of a complex between the antibody or portion thereof and human PRO-C3 and/or C4G. In some aspects, the test and control tissue samples are FFPE samples. The formation of a complex is then detected, wherein a difference in complex formation between the test sample and the negative control sample is indicative of the presence of human PRO-C3 and/or C4G antigen in the sample. Various methods are used to quantify PRO-C3 and/or C4G expression.

In some aspects, the automated IHC method comprises: (a) deparaffinizing and rehydrating mounted tissue sections in an autostainer; (b) retrieving antigen in an autostainer; (c) setting up reagents on an autostainer; and (d) running the autostainer to include steps of neutralizing endogenous peroxidase in the tissue specimen; blocking non-specific protein binding sites on the slides; incubating the slides with primary Ab; incubating with a postprimary blocking agent; incubating with a postprimary antibody detection agent, such as another antibody that may or may not be conjugated to a detection enzyme; incubating with a polymeric-enzyme detection reagent; adding a chromogen substrate and developing; and counterstaining with hematoxylin. In some aspects, the retrieving antigen comprises using any heat based antigen retrieval device.

### Treatment Methods

In some aspects, provided herein is a method of treating cancer comprising: (a) administering an anti-Claudin-1 antibody to a subject; (b) determining a level of PRO-C3 in a test sample from the subject; and (c) comparing the level of PRO-C3 in the test sample to a level of PRO-C3 in a control sample. In some aspects, if the level of PRO-C3 in the test sample is not decreased relative to the level of PRO-C3 in the control sample, then the dose of the anti-Claudin-1 antibody being administered to the subject is increased. In some aspects, if the level of PRO-C3 in the test sample is decreased relative to the control sample, then the dose of the anti-Claudin-1 antibody being administered to the subject is not increased. In some aspects, if the level of PRO-C3 in the test sample is decreased relative to the control sample, then the dose of the anti-Claudin-1 antibody being administered to the subject is not increased, but the subject is monitored. Monitoring a subject may include, for example, regular examinations, further measurements of the level of PRO-C3, or observation of the tumor using imaging, such as PET or CT scanning. In some aspects, provided herein is a method of treating cancer in a subject. In some aspects, the subject has been determined to have a level of PRO-C3 in a test sample from the subject that is not decreased relative to a level of PRO-C3 in a control sample. In some aspects, the subject has previously been administered an anti-Claudin-1 treatment (e.g., an anti-Claudin-1 antibody or a Claudin-1 targeting CAR disclosed herein), the method comprising administering an increased dose of the anti-Claudin-1 treatment to the subject.

In some aspects, provided herein is a method of monitoring cancer progression in a subject comprising: (a) quantifying a level of PRO-C3 in a test sample from the subject, wherein the subject has previously been administered an anti-Claudin-1 treatment; and (b) comparing the level of PRO-C3 in the test sample to a level of PRO-C3 in a control sample. In some aspects, a level of PRO-C3 in the test sample that is not decreased relative to the level of PRO-C3 in the control sample indicates that the subject is susceptible to an increased dose of the anti-Claudin-1 treatment. In some aspects, if the subject is not susceptible to an increased dose of the anti-Claudin-1 antibody, the subject is monitored. Monitoring a subject may include, for example, regular examinations, further measurements of the level of PRO-C3, or observation of the tumor using imaging, such as PET or CT scanning.

In some aspects, provided herein is a method of monitoring cancer progression in a subject comprising: (a) determining a level of PRO-C3 in a test sample from the subject; and (b) comparing the level of PRO-C3 in the test sample to a level of PRO-C3 in a control sample. In some aspects, the subject has previously been administered an anti-Claudin-1 antibody. In some aspects, a level of PRO-C3 in the test sample that is not decreased relative to the level of PROC3 in the control sample indicates that the subject is susceptible to an increased dose of the anti-Claudin-1 antibody. In some aspects, if the subject is not susceptible to an increased dose of the anti-Claudin-1 antibody, the subject is monitored. Monitoring a subject may include, for example, regular examinations, further measurements of the level of PRO-C3, or observation of the tumor using imaging, such as PET or CT scanning.

In some aspects, provided herein is a method for treating a subject suffering from cancer, the method comprising the steps of: determining whether the subject has decreased levels of PRO-C3 by obtaining a test sample from the subject and comparing the level of PRO-C3 in the test sample to a control sample. In some aspects, the subject has previously been administered a dose of an anti-Claudin-1 antibody. In some aspects, if the subject has decreased levels of PRO-C3 in the test sample relative to the control sample, then the subject is not administered an increased dose of the anti-Claudin-1 antibody. In some aspects, if the subject does not have decreased levels of PRO-C3 in the test sample relative to the control sample, then the subject is administered an increased dose of the anti-Claudin-1 antibody. In some aspects, if the subject has decreased levels of PRO-C3 in the test sample relative to the control sample, then the subject is not administered an increased dose of the anti-Claudin-1 antibody, but the subject is monitored. Monitoring a subject may include, for example, regular examinations, further measurements of the level of PRO-C3, or observation of the tumor using imaging, such as PET or CT scanning.

In some aspects, provided herein is a method for designing a customized therapy for a subject suffering from cancer which comprises (a) quantifying the level of PRO-C3 in a test sample from the subject; (b) comparing the level of PRO-C3 in the test sample obtained in step (a) with the level of PRO-C3 in a control sample; (c) determining that the subject has a not decreased level of PRO-C3 with respect to the level of PRO-C3 in the control sample; and (d) administering an increased dose of the anti-Claudin-1 antibody. In some aspects, the subject has previously been administered a dose of an anti-Claudin-1 antibody. In some aspects, if the level of PRO-C3 in the test sample obtained in step (a) is not decreased relative to the level of PROC3 in the control sample, then the subject is suitable to receive an increased dose of the anti-Claudin-1 antibody. In some aspects, if the subject is not suitable to receive an increased dose of the anti-Claudin-1 antibody, the subject is monitored. Monitoring a subject may include, for example, regular examinations, further measurements of the level of PRO-C3, or observation of the tumor using imaging, such as PET or CT scanning.

In some aspects, provided herein is a method of classifying a subject suffering from cancer into a cohort and treating said subject, comprising (a) quantifying the level of PRO-C3 in a test sample from the subject; (b) comparing the level of PRO-C3 in the test sample obtained in step (a) with the level of PRO-C3 in a control sample; (c) determining that the subject has a not decreased level of PRO-C3 with respect to the level of PRO-C3 in the control sample; (d) classifying said subject into a cohort based on the level of PRO-C3 in the test sample relative to the control sample; and (e) administering an increased dose of the anti-Claudin-1 antibody. In some aspects, the subject has previously been administered a dose of an anti-Claudin-1 antibody. In some aspects, if the level of PRO-C3 in the test sample obtained in step (a) is not decreased relative to the level of PRO-C3 in the control sample, then the subject is suitable to receive an increased dose of the anti-Claudin-1 antibody. In some aspects, if the subject is not suitable to receive an increased dose of the anti-Claudin-1 antibody, the subject is monitored. Monitoring a subject may include, for example, regular examinations, further measurements of the level of PRO-C3, or observation of the tumor using imaging, such as PET or CT scanning.

In some aspects, provided herein is a method of treating cancer comprising: (a) administering an anti-Claudin-1 antibody to a subject; (b) determining a level of C4G in a test sample from the subject; and (c) comparing the level of C4G in the test sample to a level of C4G in a control sample. In some aspects, if the level of C4G in the test sample is not increased relative to the level of C4G in the control sample, then the dose of the anti-Claudin-1 antibody being administered to the subject is increased. In some aspects, if the level of C4G in the test sample is increased relative to the control sample, then the dose of the anti-Claudin-1 antibody being administered to the subject is not increased. In some aspects, if the level of C4G in the test sample is increased relative to the control sample, then the dose of the anti-Claudin-1 antibody being administered to the subject is not increased, but the subject is monitored. Monitoring a subject may include, for example, regular examinations, further measurements of the level of C4G, or observation of the tumor using imaging, such as PET or CT scanning.

In some aspects, provided herein is a method of treating cancer in a subject that has been determined to have a level of C4G in a test sample from the subject that is not increased relative to a level of C4G in a control sample. In some aspects, the subject has previously been administered an anti-Claudin-1 treatment (e.g., an anti-Claudin-1 antibody or a Claudin-1 targeting CAR disclosed herein). In some aspects, the method comprises administering an increased dose of the anti-Claudin-1 treatment to the subject.

In some aspects, provided herein is a method of monitoring cancer progression in a subject comprising: (a) quantifying a level of C4G in a test sample from the subject; and (b) comparing the level of C4G in the test sample to a level of C4G in a control sample. In some aspects, the subject has previously been administered an anti-Claudin-1 treatment. In some aspects, a level of C4G in the test sample that is not increased relative to the level of C4G in the control sample indicates that the subject is susceptible to an increased dose of the anti-Claudin-1 treatment. In some aspects, if the subject is not susceptible to an increased dose of the anti-Claudin-1 treatment, the subject is monitored. Monitoring a subject may include, for example, regular examinations, further measurements of the level of C4G, or observation of the tumor using imaging, such as PET or CT scanning.

In some aspects, provided herein is a method of monitoring cancer progression in a subject comprising: (a) determining a level of C4G in a test sample from the subject; and (b) comparing the level of C4G in the test sample to a level of C4G in a control sample. In some aspects, the subject has previously been administered an anti-Claudin-1 antibody. In some aspects, a level of C4G in the test sample that is not increased relative to the level of C4G in the control sample indicates that the subject is susceptible to an increased dose of the anti-Claudin-1 antibody. In some aspects, if the subject is not susceptible an increased dose of the anti-Claudin-1 antibody, the subject is monitored. Monitoring a subject may include, for example, regular examinations, further measurements of the level of C4G, or observation of the tumor using imaging, such as PET or CT scanning.

In some aspects, provided herein is a method for treating a subject suffering from cancer, the method comprising the steps of: determining whether the subject has increased levels of C4G by obtaining a test sample from the subject and comparing the level of C4G in the test sample to a control sample. In some aspects, the subject has previously been administered a dose of an anti-Claudin-1 antibody. In some aspects, if the subject has increased levels of C4G in the test sample relative to the control sample, then the subject is not administered an increased dose of the anti-Claudin-1 antibody. In some aspects, if the subject does not have increased levels of C4G in the test sample relative to the control sample, then the subject is administered an increased dose of the anti-Claudin-1 antibody. In some aspects, if the subject has increased levels of C4G in the test sample relative to the control sample, then the subject is not administered an increased dose of the anti-Claudin-1 antibody, but the subject is monitored. Monitoring a subject may include, for example, regular examinations, further measurements of the level of PRO-C3, or observation of the tumor using imaging, such as PET or CT scanning.

In some aspects, provided herein is a method for designing a customized therapy for a subject suffering from cancer which comprises (a) quantifying the level of C4G in a test sample from the subject; (b) comparing the level of C4G in the test sample obtained in step (a) with the level of C4G in a control sample; (c) determining that the subject has a not increased level of C4G with respect to the level of C4G in the control sample; and (d) administering an increased dose of the anti-Claudin-1 antibody. In some aspects, the subject has previously been administered a dose of an anti-Claudin-1 antibody. In some aspects, if the level of C4G in the test sample obtained in step (a) is not increased relative to the level of C4G in the control sample, then the subject is suitable to receive an increased dose of the anti-Claudin-1 antibody. In some aspects, if the subject is not suitable to receive an increased dose of the anti-Claudin-1 antibody, the subject is monitored. Monitoring a subject may include, for example, regular examinations, further measurements of the level of C4G, or observation of the tumor using imaging, such as PET or CT scanning.

In some aspects, provided herein is a method of classifying a subject suffering from cancer into a cohort and treating said subject, comprising (a) quantifying the level of C4G in a test sample from the subject; (b) comparing the level of C4G in the test sample obtained in step (a) with the level of C4G in a control sample; (c) determining that the subject has a not increased level of C4G with respect to the level of C4G in the control sample; (d) classifying said subject into a cohort based on the level of C4G in the test sample relative to the control sample; and (e) administering an increased dose of the anti-Claudin-1 antibody. In some aspects, the subject has previously been administered a dose of an anti-Claudin-1 antibody. In some aspects, if the level of C4G in the test sample obtained in step (a) is not increased relative to the level of C4G in the control sample, then the subject is suitable to receive an increased dose of the anti-Claudin-1 antibody. In some aspects, if the subject is not suitable to receive an increased dose of the anti-Claudin-1 antibody, the subject is monitored. Monitoring a subject may include, for example, regular examinations, further measurements of the level of C4G, or observation of the tumor using imaging, such as PET or CT scanning.

In some aspects, provided herein is a method of treating cancer comprising: (a) determining a level of PRO-C3 in a test sample from the subject; and (b) comparing the level of PRO-C3 in the test sample to a level of PRO-C3 in a control sample. In some aspects, if the level of PRO-C3 in the test sample is not decreased relative to the level of PRO-C3 in the control sample, then the subject is administered a dose of the anti-Claudin-1 antibody. In some aspects, if the level of PRO-C3 in the test sample is decreased relative to the control sample, then the subject is not administered a dose of the anti-Claudin-1 antibody. In some aspects, if the level of PRO-C3 in the test sample is decreased relative to the control sample, then the subject is not administered a dose of the anti-Claudin-1 antibody, but the subject is monitored. Monitoring a subject may include, for example, regular examinations, further measurements of the level of PRO-C3, or observation of the tumor using imaging, such as PET or CT scanning.

In some aspects, provided herein is a method of treating cancer in a subject. In some aspects, the subject has been determined to have a level of PRO-C3 in a test sample from the subject that is not decreased relative to a level of PRO-C3 in a control sample. In some aspects, the method comprises administering an increased dose of the anti-Claudin-1 treatment (e.g., an anti-Claudin-1 antibody or a Claudin-1 targeting CAR disclosed herein) to the subject.

In some aspects, provided herein is a method of monitoring cancer progression in a subject comprising: (a) quantifying a level of PRO-C3 in a test sample from the subject; and (b) comparing the level of PRO-C3 in the test sample to a level of PRO-C3 in a control sample. In some aspects, a level of PRO-C3 in the test sample that is not decreased relative to the level of PRO-C3 in the control sample indicates that the subject is suitable to receive a dose of an anti-Claudin-1 treatment. In some aspects, if the subject is not suitable to receive a dose of the anti-Claudin-1 treatment, the subject is monitored. Monitoring a subject may include, for example, regular examinations, further measurements of the level of PRO-C3, or observation of the tumor using imaging, such as PET or CT scanning.

In some aspects, provided herein is a method of monitoring cancer progression in a subject comprising: (a) determining a level of PRO-C3 in a test sample from the subject; and (b) comparing the level of PRO-C3 in the test sample to a level of PRO-C3 in a control sample. In some aspects, a level of PRO-C3 in the test sample that is not decreased relative to the level of PRO-C3 in the control sample indicates that the subject is suitable to receive a dose of the anti-Claudin-1 antibody. In some aspects, if the subject is not suitable to receive a dose of the anti-Claudin-1 antibody, the subject is monitored. Monitoring a subject may include, for example, regular examinations, further measurements of the level of PRO-C3, or observation of the tumor using imaging, such as PET or CT scanning.

In some aspects, provided herein is a method for treating a subject suffering from cancer, the method comprising the steps of: determining whether the subject has decreased levels of PRO-C3 by obtaining a test sample from the subject and comparing the level of PRO-C3 in the test sample to a control sample. In some aspects, if the subject has decreased levels of PROC3 in the test sample relative to the control sample, then the subject is not administered an increased dose of the anti-Claudin-1 antibody. In some aspects, if the subject does not have decreased levels of PRO-C3 in the test sample relative to the control sample, then the subject is administered a dose of the anti-Claudin-1 antibody. In some aspects, if the subject has decreased levels of PROC3 in the test sample relative to the control sample, then the subject is not administered an increased dose of the anti-Claudin-1 antibody, but the subject is monitored. Monitoring a subject may include, for example, regular examinations, further measurements of the level of PRO-C3, or observation of the tumor using imaging, such as PET or CT scanning.

In some aspects, provided herein is a method for designing a customized therapy or a subject suffering from cancer which comprises (a) quantifying the level of PRO-3 in a test sample from the subject; (b) comparing the level of PRO-C3 in the test sample obtained in step (a) with the level of PRO-C3 in a control sample; (c) determining that the subject has a not decreased level of PRO-C3 with respect to the level of PRO-C3 in the control sample; and (d) administering a dose of the anti-Claudin-1 antibody. In some aspects, if the level of PRO-C3 in the test sample obtained in step (a) is not decreased relative to the level of PRO-C3 in the control sample, then the subject is suitable to receive a dose of the anti-Claudin-1 antibody. In some aspects, if the subject is not suitable to receive a dose of the anti-Claudin-1 antibody, the subject is monitored. Monitoring a subject may include, for example, regular examinations, further measurements of the level of PRO-C3, or observation of the tumor using imaging, such as PET or CT scanning.

In some aspects, provided herein is a method of classifying a subject suffering from cancer into a cohort and treating said subject, comprising (a) quantifying the level of PRO-C3 in a test sample from the subject; (b) comparing the level of PRO-C3 in the test sample obtained in step (a) with the level of PRO-C3 in a control sample; (c) determining that the subject has a not decreased level of PRO-C3 with respect to the level of PRO-C3 in the control sample; (d) classifying said subject into a cohort based on the level of PRO-C3 in the test sample relative to the control sample; and (e) administering a dose of the anti-Claudin-1 antibody. In some aspects, if the level of PRO-C3 in the test sample obtained in step (a) is not decreased relative to the level of PRO-C3 in the control sample, then the subject is suitable to receive a dose of the anti-Claudin-1 antibody. In some aspects, if the subject is not suitable to receive an increased dose of the anti-Claudin-1 antibody, the subject is monitored. Monitoring a subject may include, for example, regular examinations, further measurements of the level of PRO-C3, or observation of the tumor using imaging, such as PET or CT scanning.

In some aspects, provided herein is a method of treating cancer comprising: (a) determining a level of C4G in a test sample from the subject; and (b) comparing the level of C4G in the test sample to a level of C4G in a control sample. In some aspects, if the level of C4G in the test sample is not increased relative to the level of C4G in the control sample, then the subject is administered a dose of an anti-Claudin-1 antibody. In some aspects, if the level of C4G in the test sample is increased relative to the control sample, then the subject is not administered an anti-Claudin-1 antibody. In some aspects, if the level of C4G in the test sample is increased relative to the control sample, then the subject is not administered an anti-Claudin-1 antibody, but the subject is monitored. Monitoring a subject may include, for example, regular examinations, further measurements of the level of C4G, or observation of the tumor using imaging, such as PET or CT scanning.

In some aspects, provided herein is a method of treating cancer in a subject that has been determined to have a level of C4G in a test sample from the subject that is not increased relative to a level of C4G in a control sample. In some aspects, the method comprises administering a dose of an anti-Claudin-1 treatment (e.g., an anti-Claudin-1 antibody or a Claudin-1 targeting CAR disclosed herein) to the subject.

In some aspects, provided herein is a method of monitoring cancer progression in a subject comprising: (a) quantifying a level of C4G in a test sample from the subject; and (b) comparing the level of C4G in the test sample to a level of C4G in a control sample. In some aspects, a level of C4G in the test sample that is not increased relative to the level of C4G in the control sample indicates that the subject is susceptible to a dose of an anti-Claudin-1 treatment. In some aspects, if the subject is not susceptible to a dose of the anti-Claudin-1 treatment, the subject is monitored. Monitoring a subject may include, for example, regular examinations, further measurements of the level of C4G, or observation of the tumor using imaging, such as PET or CT scanning.

In some aspects, provided herein is a method of monitoring cancer progression in a subject comprising: (a) determining a level of C4G in a test sample from the subject; and (b) comparing the level of C4G in the test sample to a level of C4G in a control sample. In some aspects, a level of C4G in the test sample that is not increased relative to the level of C4G in the control sample indicates that the subject is susceptible to a dose of an anti-Claudin-1 antibody. In some aspects, if the subject is not susceptible to a dose of the anti-Claudin-1 antibody, the subject is monitored. Monitoring a subject may include, for example, regular examinations, further measurements of the level of C4G, or observation of the tumor using imaging, such as PET or CT scanning.

In some aspects, provided herein is a method for treating a subject suffering from cancer, the method comprising the steps of: determining whether the subject has increased levels of C4G by obtaining a test sample from the subject and comparing the level of C4G in the test sample to a control sample. In some aspects, if the subject has increased levels of C4G in the test sample relative to the control sample, then the subject is not administered a dose of an anti-Claudin-1 antibody. In some aspects, if the subject does not have increased levels of C4G in the test sample relative to the control sample, then the subject is administered a dose of an anti-Claudin-1 antibody. In some aspects, if the subject has increased levels of C4G in the test sample relative to the control sample, then the subject is not administered a dose of an anti-Claudin-1 antibody, but the subject is monitored. Monitoring a subject may include, for example, regular examinations, further measurements of the level of C4G, or observation of the tumor using imaging, such as PET or CT scanning

In some aspects, provided herein is a method for designing a customized therapy for a subject suffering from cancer which comprises (a) quantifying the level of C4G in a test sample from the subject; (b) comparing the level of C4G in the test sample obtained in step (a) with the level of C4G in a control sample; (c) determining that the subject has a not increased level of C4G with respect to the level of C4G in the control sample; and (d) administering a dose of an anti-Claudin-1 antibody. In some aspects, if the level of C4G in the test sample obtained in step (a) is not increased relative to the level of C4G in the control sample, then the subject is suitable to receive a dose of the anti-Claudin-1 antibody. In some aspects, if the subject is not suitable to receive a dose of the anti-Claudin-1 antibody, the subject is monitored. Monitoring a subject may include, for example, regular examinations, further measurements of the level of C4G, or observation of the tumor using imaging, such as PET or CT scanning.

In some aspects, provided herein is a method of classifying a subject suffering from cancer into a cohort and treating said subject, comprising (a) quantifying the level of C4G in a test sample from the subject; (b) comparing the level of C4G in the test sample obtained in step (a) with the level of C4G in a control sample; (c) determining that the subject has a not increased level of C4G with respect to the level of C4G in the control sample; (d) classifying said subject into a cohort based on the level of C4G in the test sample relative to the control sample; and (e) administering a dose of an anti-Claudin-1 antibody. In some aspects, if the level of C4G in the test sample obtained in step (a) is not increased relative to the level of C4G in the control sample, then the subject is suitable to receive a dose of an anti-Claudin-1 antibody. In some aspects, if the subject is not suitable to receive a dose of the anti-Claudin-1 antibody, the subject is monitored. Monitoring a subject may include, for example, regular examinations, further measurements of the level of C4G, or observation of the tumor using imaging, such as PET or CT scanning.

In some aspects, provided herein is a method of treating cancer comprising: (a) administering an anti-Claudin-1 antibody to a subject; (b) determining the levels of PRO-C3 and C4G in a test sample from the subject; and (c) comparing the levels of PRO-C3 and C4G in the test sample to the levels of PRO-C3 and C4G in a control sample. In some aspects, if the levels of PRO-C3 and C4G in the test sample are not decreased relative to the levels of PRO-C3 and C4G in the control sample, then the dose of the anti-Claudin-1 antibody being administered to the subject is increased. In some aspects, if the levels of PRO-C3 and C4G in the test sample are not decreased relative to the levels of PRO-C3 and C4G in the control sample, then the dose of the anti-Claudin-1 antibody being administered to the subject is not increased. In some aspects, if the levels of PRO-C3 and C4G in the test sample are not decreased relative to the levels of PRO-C3 and C4G in the control sample, then the dose of the anti-Claudin-1 antibody being administered to the subject is not increased, but the subject is monitored. Monitoring a subject may include, for example, regular examinations, further measurements of the level of PRO-C3 and C4G, or observation of the tumor using imaging, such as PET or CT scanning.

In some aspects, provided herein is a method of treating cancer in a subject. In some aspects, the subject has been determined to have levels of PRO-C3 and C4G in a test sample from the subject that are not decreased relative to the levels of PRO-C3 and C4G in a control sample. In some aspects, the subject has previously been administered an anti-Claudin-1 treatment (e.g., an anti-Claudin-1 antibody or a Claudin-1 targeting CAR disclosed herein), the method comprising administering an increased dose of the anti-Claudin-1 treatment to the subject.

In some aspects, provided herein is a method of monitoring cancer progression in a subject comprising: (a) quantifying the levels of PRO-C3 and C4G in a test sample from the subject, wherein the subject has previously been administered an anti-Claudin-1 treatment; and (b) comparing the levels of PRO-C3 and C4G in the test sample to the levels of PRO-C3 and C4G in a control sample. In some aspects, levels of PRO-C3 and C4G in the test sample that are not decreased relative to the level of PRO-C3 and C4G in the control sample indicates that the subject is susceptible to an increased dose of the anti-Claudin-1 treatment. In some aspects, if the subject is not susceptible to an increased dose of the anti-Claudin-1 antibody, the subject is monitored. Monitoring a subject may include, for example, regular examinations, further measurements of the level of PRO-C3 and C4G, or observation of the tumor using imaging, such as PET or CT scanning.

In some aspects, provided herein is a method of monitoring cancer progression in a subject comprising: (a) determining the levels of PRO-C3 and C4G in a test sample from the subject; and (b) comparing the levels of PRO-C3 and C4G in the test sample to the levels of PRO-C3 and C4G in a control sample. In some aspects, the subject has previously been administered an anti-Claudin-1 antibody. In some aspects, levels of PRO-C3 and C4G in the test sample that are not decreased relative to the levels of PROC3 and C4G in the control sample indicates that the subject is susceptible to an increased dose of the anti-Claudin-1 antibody. In some aspects, if the subject is not susceptible to an increased dose of the anti-Claudin-1 antibody, the subject is monitored. Monitoring a subject may include, for example, regular examinations, further measurements of the level of PROC3 and C4G, or observation of the tumor using imaging, such as PET or CT scanning.

In some aspects, provided herein is a method for treating a subject suffering from cancer, the method comprising the steps of: determining whether the subject has decreased levels of PRO-C3 and C4G by obtaining a test sample from the subject and comparing the levels of PRO-C3 and C4G in the test sample to a control sample. In some aspects, the subject has previously been administered a dose of an anti-Claudin-1 antibody. In some aspects, if the subject has decreased levels of PRO-C3 and C4G in the test sample relative to the control sample, then the subject is not administered an increased dose of the anti-Claudin-1 antibody. In some aspects, if the subject does not have decreased levels of PRO-C3 and C4G in the test sample relative to the control sample, then the subject is administered an increased dose of the anti-Claudin-1 antibody. In some aspects, if the subject has decreased levels of PRO-C3 and C4G in the test sample relative to the control sample, then the subject is not administered an increased dose of the anti-Claudin-1 antibody, but the subject is monitored. Monitoring a subject may include, for example, regular examinations, further measurements of the level of PRO-C3 and C4G, or observation of the tumor using imaging, such as PET or CT scanning.

In some aspects, provided herein is a method for designing a customized therapy for a subject suffering from cancer which comprises (a) quantifying the levels of PRO-C3 and C4G in a test sample from the subject; (b) comparing the levels of PRO-C3 and C4G in the test sample obtained in step (a) with the levels of PRO-C3 and C4G in a control sample; (c) determining that the subject does not have decreased levels of PRO-C3 and C4G with respect to the level of PRO-C3 and C4G in the control sample; and (d) administering an increased dose of the anti-Claudin-1 antibody. In some aspects, the subject has previously been administered a dose of an anti-Claudin-1 antibody. In some aspects, if the levels of PRO-C3 and C4G in the test sample obtained in step (a) is not decreased relative to the levels of PROC3 and C4G in the control sample, then the subject is suitable to receive an increased dose of the anti-Claudin-1 antibody. In some aspects, if the subject is not suitable to receive an increased dose of the anti-Claudin-1 antibody, the subject is monitored. Monitoring a subject may include, for example, regular examinations, further measurements of the level of PRO-C3 and C4G, or observation of the tumor using imaging, such as PET or CT scanning.

In some aspects, provided herein is a method of classifying a subject suffering from cancer into a cohort and treating said subject, comprising (a) quantifying the levels of PRO-C3 and C4G in a test sample from the subject; (b) comparing the levels of PRO-C3 and C4G in the test sample obtained in step (a) with the levels of PRO-C3 and C4G in a control sample; (c) determining that the subject does not have decreased levels of PRO-C3 and C4G with respect to the levels of PRO-C3 and C4G in the control sample; (d) classifying said subject into a cohort based on the levels of PRO-C3 and C4G in the test sample relative to the control sample; and (e) administering an increased dose of the anti-Claudin-1 antibody. In some aspects, the subject has previously been administered a dose of an anti-Claudin-1 antibody. In some aspects, if the levels of PRO-C3 and C4G in the test sample obtained in step (a) are not decreased relative to the levels of PRO-C3 and C4G in the control sample, then the subject is suitable to receive an increased dose of the anti-Claudin-1 antibody. In some aspects, if the subject is not suitable to receive an increased dose of the anti-Claudin-1 antibody, the subject is monitored. Monitoring a subject may include, for example, regular examinations, further measurements of the level of PRO-C3 and C4G, or observation of the tumor using imaging, such as PET or CT scanning.

In some aspects, provided herein is a method of treating cancer comprising: (a) determining the levels of PRO-C3 and C4G in a test sample from the subject; and (b) comparing the levels of PRO-C3 and C4G in the test sample to the levels of PRO-C3 and C4G in a control sample. In some aspects, if the levels of PRO-C3 and C4G in the test sample is not decreased relative to the levels of PRO-C3 and C4G in the control sample, then the subject is administered a dose of the anti-Claudin-1 antibody. In some aspects, if the levels of PRO-C3 and C4G in the test sample are decreased relative to the control sample, then the subject is not administered a dose of the anti-Claudin-1 antibody. In some aspects, if the levels of PRO-C3 and C4G in the test sample are decreased relative to the control sample, then the subject is not administered a dose of the anti-Claudin-1 antibody, but the subject is monitored. Monitoring a subject may include, for example, regular examinations, further measurements of the level of PRO-C3 and C4G, or observation of the tumor using imaging, such as PET or CT scanning.

In some aspects, provided herein is a method of treating cancer in a subject. In some aspects, the subject has been determined to have levels of PRO-C3 and C4G in a test sample from the subject that are not decreased relative to the levels of PRO-C3 and C4G in a control sample. In some aspects, the method comprises administering an increased dose of the anti-Claudin-1 treatment (e.g., an anti-Claudin-1 antibody or a Claudin-1 targeting CAR disclosed herein) to the subject.

In some aspects, provided herein is a method of monitoring cancer progression in a subject comprising: (a) quantifying the levels of PRO-C3 and C4G in a test sample from the subject; and (b) comparing the levels of PRO-C3 and C4G in the test sample to the levels of PRO-C3 and C4G in a control sample. In some aspects, levels of PRO-C3 and C4G in the test sample that are not decreased relative to the levels of PRO-C3 and C4G in the control sample indicate that the subject is suitable to receive a dose of an anti-Claudin-1 treatment. In some aspects, if the subject is not suitable to receive a dose of the anti-Claudin-1 treatment, the subject is monitored. Monitoring a subject may include, for example, regular examinations, further measurements of the level of PRO-C3 and C4G, or observation of the tumor using imaging, such as PET or CT scanning.

In some aspects, provided herein is a method of monitoring cancer progression in a subject comprising: (a) determining the levels of PRO-C3 and C4G in a test sample from the subject; and (b) comparing the levels of PRO-C3 and C4G in the test sample to the levels of PRO-C3 and C4G in a control sample. In some aspects, levels of PRO-C3 and C4G in the test sample that are not decreased relative to the levels of PRO-C3 and C4G in the control sample indicate that the subject is suitable to receive a dose of the anti-Claudin-1 antibody. In some aspects, if the subject is not suitable to receive a dose of the anti-Claudin-1 antibody, the subject is monitored. Monitoring a subject may include, for example, regular examinations, further measurements of the level of PRO-C3 and C4G, or observation of the tumor using imaging, such as PET or CT scanning.

In some aspects, provided herein is a method for treating a subject suffering from cancer, the method comprising the steps of: determining whether the subject has decreased levels of PRO-C3 and C4G by obtaining a test sample from the subject and comparing the levels of PRO-C3 and C4G in the test sample to a control sample. In some aspects, if the subject has decreased levels of PROC3 and C4G in the test sample relative to the control sample, then the subject is not administered an increased dose of the anti-Claudin-1 antibody. In some aspects, if the subject does not have decreased levels of PRO-C3 and C4G in the test sample relative to the control sample, then the subject is administered a dose of the anti-Claudin-1 antibody. In some aspects, if the subject has decreased levels of PROC3 and C4G in the test sample relative to the control sample, then the subject is not administered an increased dose of the anti-Claudin-1 antibody, but the subject is monitored. Monitoring a subject may include, for example, regular examinations, further measurements of the level of PRO-C3 and C4G, or observation of the tumor using imaging, such as PET or CT scanning.

In some aspects, provided herein is a method for designing a customized therapy or a subject suffering from cancer which comprises (a) quantifying the levels of PRO-C3 and C4G in a test sample from the subject; (b) comparing the levels of PRO-C3 and C4G in the test sample obtained in step (a) with the levels of PRO-C3 and C4G in a control sample; (c) determining that the subject does not have decreased levels of PRO-C3 and C4G with respect to the level of PRO-C3 and C4G in the control sample; and (d) administering a dose of the anti-Claudin-1 antibody. In some aspects, if the levels of PRO-C3 and C4G in the test sample obtained in step (a) are not decreased relative to the levels of PRO-C3 and C4G in the control sample, then the subject is suitable to receive a dose of the anti-Claudin-1 antibody. In some aspects, if the subject is not suitable to receive a dose of the anti-Claudin-1 antibody, the subject is monitored. Monitoring a subject may include, for example, regular examinations, further measurements of the level of PRO-C3 and C4G, or observation of the tumor using imaging, such as PET or CT scanning.

In some aspects, provided herein is a method of classifying a subject suffering from cancer into a cohort and treating said subject, comprising (a) quantifying the levels of PRO-C3 and C4G in a test sample from the subject; (b) comparing the levels of PRO-C3 and C4G in the test sample obtained in step (a) with the levels of PRO-C3 and C4G in a control sample; (c) determining that the subject does not have decreased levels of PRO-C3 and C4G with respect to the levels of PRO-C3 and C4G in the control sample; (d) classifying said subject into a cohort based on the levels of PRO-C3 and C4G in the test sample relative to the control sample; and (e) administering a dose of the anti-Claudin-1 antibody. In some aspects, if the levels of PRO-C3 and C4G in the test sample obtained in step (a) are not decreased relative to the levels of PRO-C3 and C4G in the control sample, then the subject is suitable to receive a dose of the anti-Claudin-1 antibody. In some aspects, if the subject is not suitable to receive an increased dose of the anti-Claudin-1 antibody, the subject is monitored. Monitoring a subject may include, for example, regular examinations, further measurements of the level of PRO-C3 and C4G, or observation of the tumor using imaging, such as PET or CT scanning.

In some aspects, if the subject is susceptible to an increased dose of the anti-Claudin-1 antibody, then the anti-Claudin-1 antibody is administered to the subject.

In some aspects, the anti-Claudin-1 treatment is an anti-Claudin-1 antibody.

In some aspects, the anti-Claudin-1 antibody is administered intratumorally, intravenously, intraperitoneally, intramuscularly, intrathecally or subcutaneously.

In some aspects, the cancer is selected from the group consisting of a head and neck cancer (e.g., Head and Neck Squamous Cell Carcinoma), a lung cancer, a breast cancer, a melanoma, a colorectal cancer, a pancreatic cancer, an esophageal cancer, a cholangiocarcinoma, and a hepatocellular carcinoma.

In some aspects, the control sample is a sample from the subject prior to being administered an anti-Claudin-1 antibody or an anti-Claudin-1 treatment.

In some aspects, the level of PRO-C3 in the control sample is about 15 ng/ml. In some aspects, the level of PRO-C3 in the control sample is about 10 ng/ml to about 30 ng/mL. In some aspects, the level of PRO-C3 in the control sample is about 10 ng/ml to about 25 ng/mL. In some aspects, the level of PRO-C3 in the control sample is about 10 ng/ml to about 20 ng/mL. In some aspects, the level of PRO-C3 in the control sample is about 10 ng/mL to about 15 ng/mL. In some aspects, the level of PRO-C3 in the control sample is about 15 ng/ml to about 30 ng/mL. In some aspects, the level of PRO-C3 in the control sample is about 20 ng/mL to about 30 ng/mL. In some aspects, the level of PRO-C3 in the control sample is about 25 ng/ml to about 30 ng/mL.

In some aspects, the level of PRO-C3 in the control sample is 15 ng/mL. In some aspects, the level of PRO-C3 in the control sample is 10 ng/ml to 30 ng/mL. In some aspects, the level of PRO-C3 in the control sample is 10 ng/ml to 25 ng/mL. In some aspects, the level of PRO-C3 in the control sample is 10 ng/mL to 20 ng/mL. In some aspects, the level of PRO-C3 in the control sample is 10 ng/mL to 15 ng/mL. In some aspects, the level of PRO-C3 in the control sample is 15 ng/mL to 30 ng/mL. In some aspects, the level of PRO-C3 in the control sample is 20 ng/mL to 30 ng/mL. In some aspects, the level of PRO-C3 in the control sample is 25 ng/ml to 30 ng/mL.

In some aspects, the level of C4G in the control sample is about 25 ng/ml. In some aspects, the level of C4G in the control sample is about 20 ng/ml to about 35 ng/mL. In some aspects, the level of C4G in the control sample is about 25 ng/ml to about 35 ng/ml. In some aspects, the level of C4G in the control sample is about 30 ng/mL to about 35 ng/ml. In some aspects, the level of C4G in the control sample is about 25 ng/ml to about 35 ng/ml. In some aspects, the level of C4G in the control sample is about 30 ng/ml to about 35 ng/mL. In some aspects, the level of C4G in the control sample is about 25 ng/ml to about 30 ng/mL.

In some aspects, the level of C4G in the control sample is 25 ng/mL. In some aspects, the level of C4G in the control sample is 20 ng/mL to 35 ng/mL. In some aspects, the level of C4G in the control sample is 25 ng/mL to 35 ng/mL. In some aspects, the level of C4G in the control sample is 30 ng/mL to 35 ng/mL. In some aspects, the level of C4G in the control sample is 25 ng/mL to 35 ng/mL. In some aspects, the level of C4G in the control sample is 30 ng/mL to 35 ng/mL. In some aspects, the level of C4G in the control sample is 25 ng/ml to 30 ng/mL.

In some aspects, the level of PRO-C3 in the control sample is about 15 ng/ml and the level of C4G in the control sample is about 25 ng/ml.

In some aspects, the level of PRO-C3 in the control sample is about 10 ng/ml to about 30 ng/mL and the level of C4G in the control sample is about 20 ng/mL to about 35 ng/mL. In some aspects, the level of PRO-C3 in the control sample is about 10 ng/ml to about 25 ng/mL and the level of C4G in the control sample is about 25 ng/ml to about 35 ng/mL. In some aspects, the level of PRO-C3 in the control sample is about 10 ng/ml to about 20 ng/ml and the level of C4G in the control sample is about 30 ng/mL to about 35 ng/mL. In some aspects, the level of PRO-C3 in the control sample is about 10 ng/ml to about 15 ng/ml and the level of C4G in the control sample is about 25 ng/mL to about 35 ng/mL. In some aspects, the level of PRO-C3 in the control sample is about 15 ng/ml to about 30 ng/ml and the level of C4G in the control sample is about 30 ng/mL to about 35 ng/mL. In some aspects, the level of PRO-C3 in the control sample is about 20 ng/ml to about 30 ng/ml and the level of C4G in the control sample is about 25 ng/ml to about 30 ng/mL. In some aspects, the level of PRO-C3 in the control sample is about 25 ng/ml to about 30 ng/mL and the level of C4G in the control sample is about 25 ng/ml to about 30 ng/mL.

In some aspects, the level of PRO-C3 in the control sample is 15 ng/ml and the level of C4G in the control sample is 25 ng/mL. In some aspects, the level of PRO-C3 in the control sample is 10 ng/mL to 30 ng/mL and the level of C4G in the control sample is 20 ng/mL to 35 ng/mL. In some aspects, the level of PRO-C3 in the control sample is 10 ng/ml to 25 ng/ml and the level of C4G in the control sample is 25 ng/ml to 35 ng/ml. In some aspects, the level of PRO-C3 in the control sample is 10 ng/mL to 20 ng/mL and the level of C4G in the control sample is 30 ng/mL to 35 ng/mL. In some aspects, the level of PRO-C3 in the control sample is 10 ng/mL to 15 ng/mL and the level of C4G in the control sample is 25 ng/ml to 35 ng/mL. In some aspects, the level of PRO-C3 in the control sample is 15 ng/mL to 30 ng/ml and the level of C4G in the control sample is 30 ng/ml to 35 ng/ml. In some aspects, the level of PRO-C3 in the control sample is 20 ng/ml to 30 ng/ml and the level of C4G in the control sample is 25 ng/mL to 30 ng/mL. In some aspects, the level of PRO-C3 in the control sample is 25 ng/ml to 30 ng/mL and the level of C4G in the control sample is 25 ng/ml to 30 ng/mL.

In some aspects, the anti-Claudin-1 antibody is a monoclonal antibody comprising the six complementarity determining regions (CDRs) of an anti-Claudin-1 monoclonal antibody secreted by a hybridoma cell line deposited at the DSMZ on Jul. 29, 2008 under an Accession Number DSM ACC2938.

In some aspects, the anti-Claudin-1 antibody is humanized.

In some aspects, the anti-Claudin-1 antibody comprises a VH comprising the amino acid sequence set forth in SEQ ID NO: 3 or SEQ ID NO: 13.

In some aspects, the anti-Claudin-1 antibody comprises a VL comprising the amino acid sequence set forth in SEQ ID NO: 4 or SEQ ID NO: 14.

In some aspects, the anti-Claudin-1 antibody comprises a VH comprising the amino acid sequence set forth in SEQ ID NO: 3; and a VL comprising the amino acid sequence set forth in SEQ ID NO: 4.

In some aspects, the anti-Claudin-1 antibody comprises a VH comprising the amino acid sequence set forth in SEQ ID NO: 13; and a VL comprising the amino acid sequence set forth in SEQ ID NO: 14.

In some aspects, the anti-Claudin-1 antibody comprises a complementarity determining region (CDR) H1 comprising the amino acid sequence set forth in SEQ ID NO: 5, a CDR H2 comprising the amino acid sequence set forth in SEQ ID NO: 6, and a CDR H3 comprising the amino acid sequence set forth in SEQ ID NO: 7.

In some aspects, the anti-Claudin-1 antibody comprises a complementarity determining region (CDR) L1 comprising the amino acid sequence set forth in SEQ ID NO: 8, a CDR L2 comprising the amino acid sequence Gly Ala, and a CDR L3 comprising the amino acid sequence set forth in SEQ ID NO: 10.

### Kits

The present disclosure also provides kits, or products of manufacture, comprising an anti-Claudin-1 antibody and an ELISA kit. In some aspects, the ELISA kit measures PRO-C3. In some aspects, the ELISA kit measures C4G. In some aspects, the kit further comprises a lancet and a vial for obtaining a blood sample. In some aspects, the blood sample is a blood plasma sample.

One skilled in the art will readily recognize that the anti-Claudin-1 antibody of the present disclosure, can be readily incorporated into one of the established kit formats which are well known in the art.

The following examples are illustrative and do not limit the scope of the claimed aspects.

### EXAMPLES

### Example 1. PRO-C3 and C4G are a Biomarkers for Anti-Claudin-1 Antibody Target Engagement

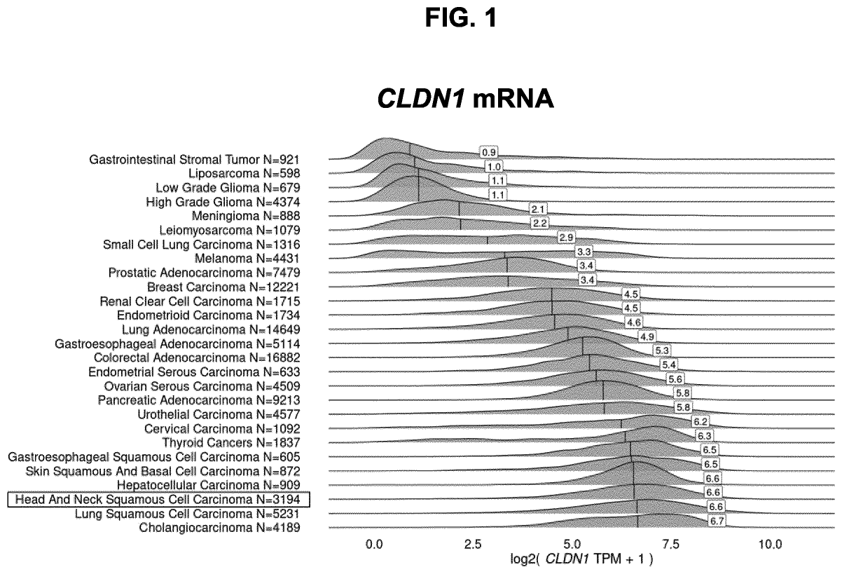

Claudin-1 (CLND1) mRNA expression was measured and shown to be expressed in numerous solid tumors from a range of cancer types (see FIG. 1).

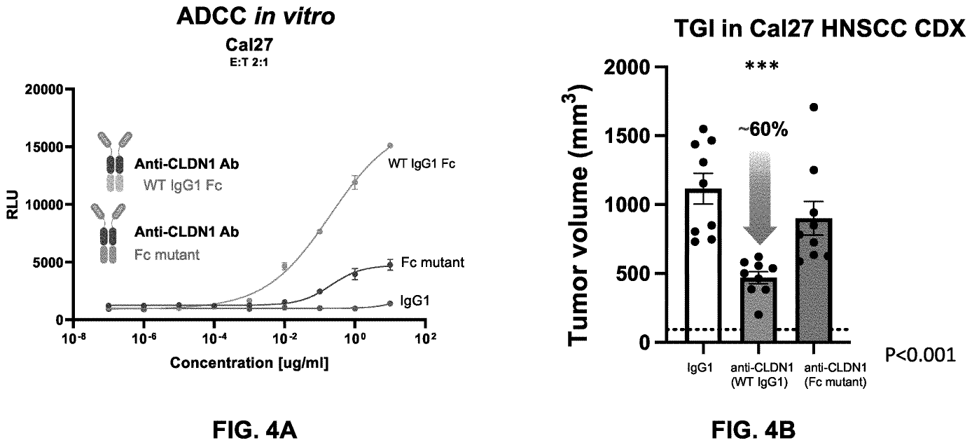

Anti-CLDN1 antibodies with a mutant Fc region, as well as anti-CLDN1 antibodies with a WT IgG1 Fc region were analyzed. The anti-CLDN1 Ab with the WT IgG1 Fc had better ADCC values as measured in vitro by relative light units (RLU), as well as better tumor growth inhibition in Cal27 HNSCC CDX model as measured by tumor volume (mm3) (see FIGS. 4A-4B).

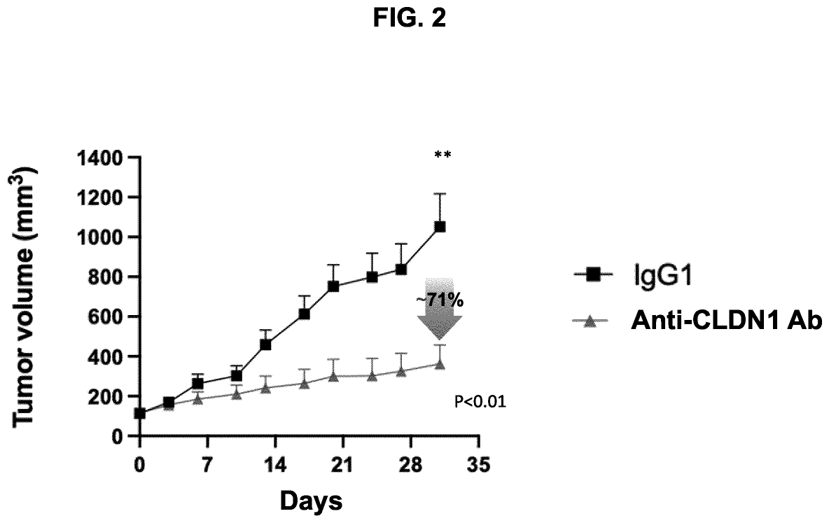

Head and Neck Squamous Cell Carcinoma (“HNSCC”) cells were used in a patient derived xenograft (“PDX”) model to measure the effectiveness of an anti-CLDN1 antibody. Tumor volume in the HNSCC PDX model decreased after treatment of anti-CLDN1 antibody as compared to treatment with an IgG1 control (see FIG. 2).

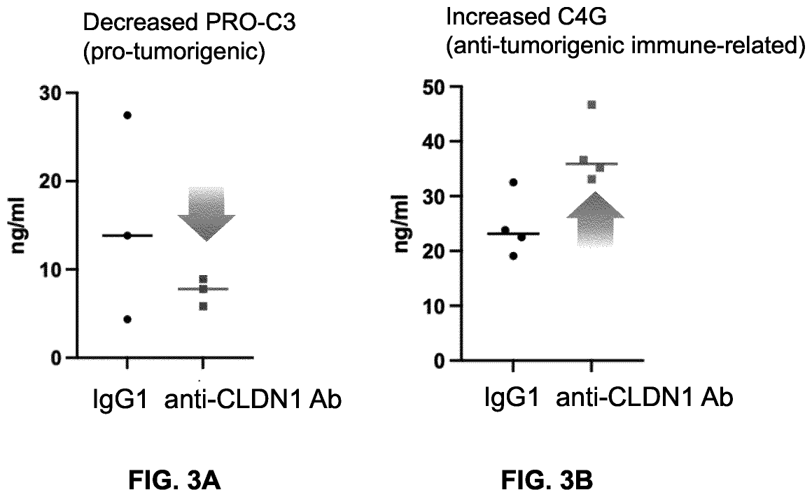

Next, Extracellular Matrix Remodeling Markers PRO-C3 and C4G were quantified in the HNSCC PDX model after treatment with an anti-CLDN1 antibody or IgG1 control. After treatment with the anti-CLDN1 antibody, PRO-C3 levels decreased and C4G levels increased. Therefore, PRO-C3 and C4G represent viable biomarkers for measuring the effectiveness of the anti-CLDN1 antibody treatment (see FIGS. 3A-3B).

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature.

All of the references cited above, as well as all references cited herein, are incorporated herein by reference in their entireties.

What is claimed is:

1. A method of treating cancer comprising:
(a) administering an anti-Claudin-1 antibody to a subject;
(b) determining a level of N-terminal propeptide of type III collagen (“PRO-C3”) in a test sample from the subject; and
(c) comparing the level of PRO-C3 in the test sample to a level of PRO-C3 in a control sample; wherein if the level of PRO-C3 in the test sample is not decreased relative to the level of PRO-C3 in the control sample, then the dose of the anti-Claudin-1 antibody being administered to the subject is increased; wherein if the level of PRO-C3 in the test sample is decreased relative to the control sample, then the dose of the anti-Claudin-1 antibody being administered to the subject is not increased.

2. A method of treating cancer in a subject, wherein the subject has been determined to have a level of PRO-C3 in a test sample from the subject that is not decreased relative to a level of PRO-C3 in a control sample, wherein the subject has previously been administered an anti-Claudin-1 treatment, the method comprising administering an increased dose of the anti-Claudin-1 treatment to the subject.

3. A method of monitoring cancer progression in a subject comprising:
(a) quantifying a level of PRO-C3 in a test sample from the subject, wherein the subject has previously been administered an anti-Claudin-1 treatment; and
(b) comparing the level of PRO-C3 in the test sample to a level of PRO-C3 in a control sample;
wherein a level of PRO-C3 in the test sample that is not decreased relative to the level of PRO-C3 in the control sample indicates that the subject is susceptible to an increased dose of the anti-Claudin-1 treatment.

4. A method of monitoring cancer progression in a subject comprising:
(a) determining a level of PRO-C3 in a test sample from the subject, wherein the subject has previously been administered an anti-Claudin-1 antibody; and
(b) comparing the level of PRO-C3 in the test sample to a level of PRO-C3 in a control sample;
wherein a level of PRO-C3 in the test sample that is not decreased relative to the level of PRO-C3 in the control sample indicates that the subject is susceptible to an increased dose of the anti-Claudin-1 antibody.

5. A method for treating a subject suffering from cancer, the method comprising the steps of:
determining whether the subject has increased levels of PRO-C3 by obtaining a test sample from the subject and comparing the level of PRO-C3 in the test sample to a control sample, wherein the subject has previously been administered a dose of an anti-Claudin-1 antibody, wherein if the subject has decreased levels of PRO-C3 in the test sample relative to the control sample, then the subject is not administered an increased dose of the anti-Claudin-1 antibody;
wherein if the subject does not have decreased levels of PRO-C3 in the test sample relative to the control sample, then the subject is administered an increased dose of the anti-Claudin-1 antibody.

6. A method for designing a customized therapy for a subject suffering from cancer which comprises
(a) quantifying the level of PRO-C3 in a test sample from the subject, wherein the subject has previously been administered a dose of an anti-Claudin-1 antibody;
(b) comparing the level of PRO-C3 in the test sample obtained in step (a) with the level of PRO-C3 in a control sample, wherein if the level of PRO-C3 in the test sample obtained in step (a) is not decreased relative to the level of PRO-C3 in the control sample, then the subject is susceptible to receive an increased dose of the anti-Claudin-1 antibody;
(c) determining that the subject has a not decreased level of PRO-C3 with respect to the level of PRO-C3 in the control sample; and
(d) administering an increased dose of the anti-Claudin-1 antibody.

7. A method of classifying a subject suffering from cancer into a cohort and treating said subject, comprising
(a) quantifying the level of PRO-C3 in a test sample from the subject, wherein the subject has previously been administered a dose of an anti-Claudin-1 antibody;
(b) comparing the level of PRO-C3 in the test sample obtained in step (a) with the level of PRO-C3 in a control sample, wherein if the level of PRO-C3 in the test sample obtained in step (a) is not decreased relative to the level of PRO-C3 in the control sample, then the subject is susceptible to receive an increased dose of the anti-Claudin-1 antibody;
(c) determining that the subject has a not decreased level of PRO-C3 with respect to the level of PRO-C3 in the control sample;
(d) classifying said subject into a cohort based on the level of PRO-C3 in the test sample relative to the control sample; and
(e) administering an increased dose of the anti-Claudin-1 antibody.

8. A method of treating cancer comprising:
(a) administering an anti-Claudin-1 antibody to a subject;
(b) determining a level of granzyme B degraded type IV collagen products (“C4G”) in a test sample from the subject; and
(c) comparing the level of C4G in the test sample to a level of C4G in a control sample;
wherein if the level of C4G in the test sample is not increased relative to the level of C4G in the control sample, then the dose of the anti-Claudin-1 antibody being administered to the subject is increased; wherein if the level of C4G in the test sample is increased relative to the control sample, then the dose of the anti-Claudin-1 antibody being administered to the subject is not increased.

9. A method of treating cancer in a subject, wherein the subject has been determined to have a level of C4G in a test sample from the subject that is not increased relative to a level of C4G in a control sample, wherein the subject has previously been administered an anti-Claudin-1 treatment, the method comprising administering an increased dose of the anti-Claudin-1 treatment to the subject.

10. A method of monitoring cancer progression in a subject comprising:
(a) quantifying a level of C4G in a test sample from the subject, wherein the subject has previously been administered an anti-Claudin-1 treatment; and
(b) comparing the level of C4G in the test sample to a level of C4G in a control sample;
wherein a level of C4G in the test sample that is not increased relative to the level of C4G in the control sample indicates that the subject is susceptible to an increased dose of the anti-Claudin-1 treatment.

11. A method of monitoring cancer progression in a subject comprising:
(a) determining a level of C4G in a test sample from the subject, wherein the subject has previously been administered an anti-Claudin-1 antibody; and
(b) comparing the level of C4G in the test sample to a level of C4G in a control sample;
wherein a level of C4G in the test sample that is not increased relative to the level of C4G in the control sample indicates that the subject is susceptible to an increased dose of the anti-Claudin-1 antibody.

12. A method for treating a subject suffering from cancer, the method comprising the steps of:
determining whether the subject has increased levels of C4G by obtaining a test sample from the subject and comparing the level of C4G in the test sample to a control sample, wherein the subject has previously been administered a dose of an anti-Claudin-1 antibody, wherein if the subject has increased levels of C4G in the test sample relative to the control sample, then the subject is not administered an increased dose of the anti-Claudin-1 antibody;
wherein if the subject does not have increased levels of C4G in the test sample relative to the control sample, then the subject is administered an increased dose of the anti-Claudin-1 antibody.

13. A method for designing a customized therapy for a subject suffering from cancer which comprises
(a) quantifying the level of C4G in a test sample from the subject, wherein the subject has previously been administered a dose of an anti-Claudin-1 antibody;
(b) comparing the level of C4G in the test sample obtained in step (a) with the level of C4G in a control sample, wherein if the level of C4G in the test sample obtained in step (a) is not increased relative to the level of C4G in the control sample, then the subject is susceptible to receive an increased dose of the anti-Claudin-1 antibody;
(c) determining that the subject has a not increased level of C4G with respect to the level of C4G in the control sample; and
(d) administering an increased dose of the anti-Claudin-1 antibody.

14. A method of classifying a subject suffering from cancer into a cohort and treating said subject, comprising
(a) quantifying the level of C4G in a test sample from the subject, wherein the subject has previously been administered a dose of an anti-Claudin-1 antibody;
(b) comparing the level of C4G in the test sample obtained in step (a) with the level of C4G in a control sample, wherein if the level of C4G in the test sample obtained in step (a) is not increased relative to the level of C4G in the control sample, then the subject is susceptible to receive an increased dose of the anti-Claudin-1 antibody;
(c) determining that the subject has a not increased level of C4G with respect to the level of C4G in the control sample;
(d) classifying said subject into a cohort based on the level of C4G in the test sample relative to the control sample; and
(e) administering an increased dose of the anti-Claudin-1 antibody.

15. The method of any one of claim 3, 4, 10, or 11, wherein if the subject is susceptible to an increased dose of the anti-Claudin-1 antibody, then the anti-Claudin-1 antibody is administered to the subject.

16. The method of any one of claim 2, 3, 9, or 10, wherein the anti-Claudin-1 treatment is an anti-Claudin-1 antibody.

17. The method of any one of claims 1-16, wherein the anti-Claudin-1 antibody is administered intratumorally, intravenously, intraperitoneally, intramuscularly, intrathecally or subcutaneously.

18. The method of any one of claims 1-17, wherein the cancer is selected from the group consisting of a head and neck cancer (e.g., Head and Neck Squamous Cell Carcinoma), a lung cancer, a breast cancer, a melanoma, a colorectal cancer, a pancreatic cancer, an esophageal cancer, a cholangiocarcinoma, and a hepatocellular carcinoma.

19. The method of any one of claims 1-18, wherein the control sample is a sample from the subject prior to being administered an anti-Claudin-1 antibody or an anti-Claudin-1 treatment.

20. The method of any one of claim 1-8, or 15-19, wherein the level of PRO-C3 in the control sample is about 10 ng/mL to about 30 ng/mL.

21. The method of any one of claims 9-19, wherein the level of C4G in the control sample is about 20 ng/mL to about 35 ng/mL.

22. The method of any one of claims 1-21, wherein the anti-Claudin-1 antibody is a monoclonal antibody comprising the six complementarity determining regions (CDRs) of an anti-Claudin-1 monoclonal antibody secreted by a hybridoma cell line deposited at the DSMZ on Jul. 29, 2008 under an Accession Number DSM ACC2938.

23. The method of any one of claims 1-22, wherein the anti-Claudin-1 antibody is humanized.

24. The method of any one of claims 1-23, wherein the anti-Claudin-1 antibody comprises a VH comprising the amino acid sequence set forth in SEQ ID NO: 3 or SEQ ID NO: 13.

25. The method of any one of claims 1-24, wherein the anti-Claudin-1 antibody comprises a VL comprising the amino acid sequence set forth in SEQ ID NO: 4 or SEQ ID NO: 14.

26. The method of any one of claims 1-25, wherein the anti-Claudin-1 antibody comprises a VH comprising the amino acid sequence set forth in SEQ ID NO: 3; and a VL comprising the amino acid sequence set forth in SEQ ID NO: 4.

27. The method of any one of claims 1-26, wherein the anti-Claudin-1 antibody comprises a VH comprising the amino acid sequence set forth in SEQ ID NO: 13; and a VL comprising the amino acid sequence set forth in SEQ ID NO: 14.

28. The method of any one of claims 1-27, wherein the anti-Claudin-1 antibody comprises a complementarity determining region (CDR) H1 comprising the amino acid sequence set forth in SEQ ID NO: 5, a CDR H2 comprising the amino acid sequence set forth in SEQ ID NO: 6, and a CDR H3 comprising the amino acid sequence set forth in SEQ ID NO: 7.

29. The method of any one of claims 1-26, wherein the anti-Claudin-1 antibody comprises a complementarity determining region (CDR) L1 comprising the amino acid sequence set forth in SEQ ID NO: 8, a CDR L2 comprising the amino acid sequence GA, and a CDR L3 comprising the amino acid sequence set forth in SEQ ID NO: 10.

30. The method of any one of claims 1-29, wherein anti-Claudin-1 antibody comprises a heavy chain comprising the amino acid sequence set forth in SEQ ID NO: 1.

31. The method of any one of claims 1-30, wherein anti-Claudin-1 antibody comprises a light chain comprising the amino acid sequence set forth in SEQ ID NO: 2.


SEQUENCE LISTING (21 sequences)

SEQ ID NO: 1 - protein, 448 aa, synthetic construct
EVQLVESGGGLVKPGGSLRLSCAASGFSFSSYGMNWVRQAPGKGLEWVSSISPSGSYFYYADSVKGRFTISRDNAKNSLYLQMNSLRAEDTAVYYCARLPGFNPPFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 214 aa, synthetic construct
DIQMTQSPATLSVSPGERATLSCKASQNVGGNVDWYQWKPGQAPRLLIYGASNRYTGIPARFRGSGSGTEFTLTISSLQSEDFAVYYCLQYKNNPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 119 aa, synthetic construct
EVQLVESGGGLVKPGGSLRLSCAASGFSFSSYGMNWVRQAPGKGLEWVSSISPSGSYFYYADSVKGRFTISRDNAKNSLYLQMNSLRAEDTAVYYCARLPGFNPPFDHWGQGTLVTVSS

SEQ ID NO: 4 - protein, 107 aa, synthetic construct
DIQMTQSPATLSVSPGERATLSCKASQNVGGNVDWYQWKPGQAPRLLIYGASNRYTGIPARFRGSGSGTEFTLTISSLQSEDFAVYYCLQYKNNPWTFGQGTKVEIK

SEQ ID NO: 5 - protein, 8 aa, synthetic construct
GFSFSSYG

SEQ ID NO: 6 - protein, 8 aa, synthetic construct
ISPSGSYF

SEQ ID NO: 7 - protein, 12 aa, synthetic construct
ARLPGFNPPFDH

SEQ ID NO: 8 - protein, 6 aa, synthetic construct
QNVGGN

SEQ ID NO: 9 - skipped

SEQ ID NO: 10 - protein, 9 aa, synthetic construct
LQYKNNPWT

SEQ ID NO: 11 - protein, 448 aa, unidentified
QVQLVESGGGVVQPGRSLRLSCLGSGFSFSSYGMNWVRQAPGKGLEWVASISPSGSYFYYADSVKGRFTISRDNSKNTLYLQMTSLRAEDTAIYYCARLPGFNPPFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 214 aa, synthetic construct
DIQMTQSPSSLSASVGDRVTITCKASQNVGGNVDWYQWKPGKAPKLLIYGASNRYTGVPDRFRGSGSGTDFTLTISSLQPEDVATYYCLQYKNNPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 119 aa, synthetic construct
QVQLVESGGGVVQPGRSLRLSCLGSGFSFSSYGMNWVRQAPGKGLEWVASISPSGSYFYYADSVKGRFTISRDNSKNTLYLQMTSLRAEDTAIYYCARLPGFNPPFDHWGQGTLVTVSS

SEQ ID NO: 14 - protein, 107 aa, synthetic construct
DIQMTQSPSSLSASVGDRVTITCKASQNVGGNVDWYQWKPGKAPKLLIYGASNRYTGVPDRFRGSGSGTDFTLTISSLQPEDVATYYCLQYKNNPWTFGGGTKVEIK

SEQ ID NO: 15 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 15 aa, synthetic construct
GGGGSGGGGSGGGGS

SEQ ID NO: 17 - protein, 18 aa, synthetic construct
GSTSGSGKPGSGEGSTKG

SEQ ID NO: 18 - protein, 45 aa, synthetic construct
TTTPAPRPPTPAPTIASQPLSLRPEACRPAAGGAVHTRGLDFACD

SEQ ID NO: 19 - protein, 24 aa, synthetic construct
IYIWAPLAGTCGVLLLSLVITLYC

SEQ ID NO: 20 - protein, 42 aa, synthetic construct
KRGRKKLLYIFKQPFMRPVQTTQEEDGCSCRFPEEEEGGCEL

SEQ ID NO: 21 - protein, 448 aa, synthetic construct
EVQLVESGGGLVKPGGSLRLSCAASGFSFSSYGMNWVRQAPGKGLEWVSSISPSGSYFYYADSVKGRFTISRDNAKNSLYLQMNSLRAEDTAVYYCARLPGFNPPFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG